(19) United States
(12) Patent Application Publication
(10) Pub. No.: US 2025/0189537 A1
(43) Pub. Date: Jun. 12, 2025

(54) MULTI-FLUORESCENCE MICROFLUIDIC PLATFORM FOR EVALUATING RISKS OF SHEAR-DRIVEN ARTERIAL THROMBOSIS AND FAST ANTITHROMBOTIC DRUG SCREENING

(71) Applicant: Board of Regents, The University of Texas System, Austin, TX (US)

(72) Inventors: Yunfeng Chen, Galveston, TX (US); Misbahud Din, Galveston, TX (US); Souvik Paul, Galveston, TX (US)

(21) Appl. No.: 18/963,402

(22) Filed: Nov. 27, 2024

Related U.S. Application Data
(60) Provisional application No. 63/607,136, filed on Dec. 7, 2023.

(51) Int. Cl. G01N 33/68; G16B 25/10

(52) U.S. Cl. CPC G01N 33/6863; G16B 25/10

(57) ABSTRACT

Embodiments of the invention are directed to a thrombus profiling assay that combines high shear flow and stenotic geometries with multi-color fluorescence imaging to comprehensively characterize thrombi forming under the physiological settings of arterial thrombosis.

### RELATED APPLICATIONS

This application claims benefit of U.S. Provisional Application 63/607,136 filed Dec. 7, 2023 which is incorporated herein by reference in its entirety.

### STATEMENT REGARDING FEDERALLY FUNDED RESEARCH

This invention was made with government support under ROOHL153678 and P30-AG024832 awarded by the National Heart, Lung, and Blood Institute (NHLBI) and National Institute of Aging (NIA), respectively. The government has certain rights in the invention.

### BACKGROUND

Arterial thrombosis, which describes the formation of pathological blood clots in the artery, is one of the leading causes of mortality and morbidity worldwide (Benjamin et al. Circulation 139, e56-e528, 2019; Springer, Blood 124, 1412-1425, 2014). A symbolic feature of arterial thrombosis is large-scale platelet aggregation due to elevated shear stress generated by stenosis (Nesbitt et al. Nature medicine 15, 665-673, 2009; Jackson, Nature medicine 17, 1423-1436, 2011), which we term “biomechanical platelet aggregation” (Chen and Ju, Stroke Vasc Neurol 5, 185-197, 2020; Chen et al., Nature materials 18, 760-769, 2019). Unfortunately, current hematological function assays do not adequately consider platelet mechanobiology, and thus are incapable of evaluating thrombotic risks associated with flow disturbance and high shear stress (Zhang et al., Front Cardiovasc Med 8, 766513, 2021; Zhang et al., Frontiers in pharmacology 12, 779753, 2021). The current clinical paradigm is further challenged by the limited efficacy of conventional antiplatelet therapies, which primarily target soluble agonist-induced platelet aggregation (Jackson, Nature medicine 17, 1423-1436, 2011), but do not counter the shear stress-induced platelet responses. This gap in therapeutic coverage becomes particularly critical in the context of hypertension, diabetes, metabolic syndrome, and the aging process, which not only exacerbate thrombotic risks but also foster resistance to conventional antiplatelet treatments (Angiolillo et al., Journal of the American College of Cardiology 50, 1541-1547, 2007; Akturk et al., Clin Appl Thromb Hemost 20, 749-754, 2014; Liu et al., J Geriatr Cardiol 10, 21-27, 2013). The understanding of the interrelations between these pathological conditions and biomechanical platelet aggregation is constrained by a dearth of evidence and methodological limitations.

Standard methodologies of observing biomechanical platelet aggregation include perfusing blood over stenotic channels (Nesbitt et al. Nature medicine 15, 665-673, 2009; Chen et al., Nature materials 18, 760-769, 2019; Li et al., PloS one 9, e82493, 2014; Tovar-Lopez et al., Lab on a chip 10, 291-302, 2010) and posts (Ting et al., Nature communications 10, 1204, 2019; Ju et al., Analyst 147, 1222-1235, 2022) under high shear conditions. However, these approaches cannot provide information regarding the composition of the thrombus or the activation status of platelets. The lack of systematic characterization of biomechanical platelet aggregation hinders understanding of its in-depth mechanisms and the development of its targeting anti-thrombotic agents. For instance, previous works showed that biomechanical platelet aggregation is initiated by platelet receptor glycoprotein (GP) Iba binding to von Willebrand factor (VWF); this interaction triggers GPIba mechano-signaling and up-regulates platelet integrin αIIbβ₃ to bind to VWF and fibrinogen (Fg) and further reinforce platelet aggregation (Nesbitt et al. Nature medicine 15, 665-673, 2009; Chen et al., Nature materials 18, 760-769, 2019; Zhang and Cheng, Nature materials 18, 661-662, 2019). Nonetheless, it remains poorly understood whether VWF and Fg, and the several integrin binding sites within Fg (Sanchez-Cortes and Mrksich, Chem Biol 16, 990-1000, 2009), work synergistically to crosslink integrin αIIbβ₃ or are mutually replaceable. The mechanisms behind VWF and Fg recruitment and platelet activation in biomechanical thrombus formation, as well as how various platelet-crosslinking processes and pathological conditions mediate these mechanisms, also remain unclear.

United States Patent publication US20120058500 describes a microfluidics device to provide real time monitoring of platelet aggregation of a biological sample obtained from a subject. The device comprises a channel configured for passage of the biological sample, the channel comprising a protrusion configured to induce an upstream region of shear acceleration coupled to a downstream region of shear deceleration and defining there-between a region of peak rate of shear, the downstream region of shear deceleration defining a zone of platelet aggregation. The device further comprises a platelet detection means for detecting aggregation of platelets in the zone of aggregation as a result of passage of the biological sample through the channel. However, the '500 publication fails to characterize the composition or platelet activation status of the in vitro thrombus.

There remains a need for additional methods of characterizing and categorizing a shear stress-induced thrombus.

### SUMMARY

To overcome the challenges described above, the Inventors developed a thrombus profiling assay, combining microfluidics with multi-color fluorescent imaging, enabling a detailed characterization of thrombus size, composition, and platelet activation status. This assay delineated the differential roles of complexed platelet crosslinking molecular mechanisms in thrombosis and identified the exacerbated formation of biomechanical thrombi in the context of hypertension and aging. A treatment mismatch phenomenon, identified while studying drug-disease interactions, prompts the re-evaluation of the efficacy and safety of anti-thrombotic agents gauged by a comprehensive ‘thrombus profile’ instead of a single thrombus size metric, and in the context of pathology models. The assay's precision in identifying the effects of inhibitors, in capturing thrombus profile abnormalities in unhealthy populations, and in detecting inter-individual variations in the thrombus profile and drug efficacy, underscore its potential as a tool for anti-thrombotic drug screening, diagnosis of thrombotic risks, and personalized anti-thrombotic regimen selection. It heralds a new era in thrombosis management, where individual patients' thrombus profiles dictate personalized therapeutic decisions to optimize treatment efficacy and reduce adverse outcomes.

Embodiments of the invention can include a combination of a microfluidic-based blood assay, multi-fluorescence imaging, and advanced data analysis. It provides a new technology that can evaluate the detailed process of shear-driven thrombosis by tracking all the key molecules in real-time. As compared to previous inventions which use similar designs of microfluidic chips to only evaluate the size growth of shear-driven thrombi, the advancement of this invention is the incorporation of multi-fluorescence imaging with carefully selected biomarker-staining reagents, which can provide multi-dimensional information using the same runtime. In certain aspects the thrombus profile is a 7 marker thrombus profile or an effective bar code.

Certain embodiments are directed to methods for characterizing an in vitro thrombus comprising: (i) applying shear stress to a blood sample in vitro forming a shear stress induced thrombus, the sample being from a subject having or suspected of being at risk for pathological conditions related to thrombus formation, or optionally, the sample is contacted with a thrombus regulating agent or an agent suspected of regulating thrombus formation or thrombus composition; (ii) measuring levels of sensor targets including (a) integrin αIIbβ₃, (b) fibrinogen (Fg), (c) Von Willebrand Factor (VWF), (d) P-selectin, (e) conformationally extended integrin αlbβ₃, (f) fully activated integrin αIIbβ₃, and (g) phosphatidylserine in the shear stress induced thrombus resulting in a thrombus profile; (iii) generating an effect barcode for the pathological condition or the thrombus regulating agent based on differences in the thrombus profile relative to a reference barcode, the effect barcode and reference barcode having a column for each sensor target level in (ii) above, each column including a positive, neutral, or negative effect of the pathological condition or thrombus regulating agent based on changes in the levels of the sensor targets. Shear stress can be applied by flowing a sample through a stenotic microfluidic device. A stenotic device has a constriction designed to generate the shear stress to induce thrombus formation from a sample. The sample is a blood sample or derivate of a blood sample that maintains its thrombotic capability. The column entry indicate that the marker levels have increased, decreased, or remain unchanged compared to a standard or a reference. In certain aspect the column entry can be a bar being at the top, middle or bottom of the column representing a positive, neutral, or negative effect, respectively. In other aspects the column entry is a ‘+’ symbol, a ‘0’ or a ‘−’ symbol representing a positive, neutral, or negative effect, respectively.

Certain embodiments are directed to methods for thrombus profiling comprising, (i) applying a blood sample to a shear stress thrombosis simulation device or otherwise forming an in vitro thrombus; (ii) contacting the in vitro thrombus with a plurality of sensor agents, the sensor agents comprising a detectable label and a component specific binding moiety and quantifying binding of the plurality of sensor agents to the in vitro thrombus determining each sensor agent result; (iii) generating a thrombus profile by characterizing the binding of each sensor agent, forming a thrombus profile comprising binding results for each sensor agent in an ordered readout. The method can further include characterizing the functional effect of an anti-thrombotic agent based on changes in the thrombus profile. This characterization can include comparing a sample with or without an antithrombic agent and analyzing the result by using the thrombus profile described herein, or evaluating a thrombus profile generated with an antithrombotic agent and comparing the profile to standard of reference. The method can be used for testing blood of a subject having a predetermined thrombus or sensor agent profile for a first condition or a first anti-thrombotic agent after the subject is exposed to a second anti-thrombotic treatment or a second anti-thrombotic agent for assessing efficacy and safety of the second anti-thrombotic treatment or the second anti-thrombotic agent or for selecting an optimal treatment regimen for the subject. The binding moieties can include a (a) conformation independent integrin αubβ₃ binding moiety, (b) fibrinogen (Fg) binding moiety, (c) Von Willebrand Factor (VWF) binding moiety, (d) P-selecting binding moiety, (e) conformationally extended integrin αIIbβ₃ binding moiety, (f) fully activated integrin αIIbβ₃ binding moiety, and (g) phosphatidylserine binding moiety. In certain aspects the conformation independent αlbβ₃ binding moiety is a SZ22 antibody; the fibrinogen (Fg) binding moiety is fibrinogen; the Von Willebrand Factor (VWF) binding moiety is a 2.2.9 antibody; the P-selectin binding moiety is a AK4 antibody; the conformationally extended integrin αIIbβ₃ binding moiety is a MBC 370.2 antibody; the fully activated integrin βubβ₃ binding moiety is a PAC-1 antibody, and the phosphatidylserine binding moiety is Annexin V.

Certain embodiments are directed to methods for generating a thrombolytic profile comprising: (i) contacting an in vitro thrombus with a plurality of sensor agents, the sensor agents comprising a detectable label and a component specific binding moiety, the binding moieties comprising a (a) conformation independent integrin βubβ₃ binding moiety, (b) fibrinogen (Fg) binding moiety, (c) Von Willebrand Factor (VWF) binding moiety, (d) P-selectin binding moiety, (e) conformationally extended integrin αIIbβ₃ binding moiety, (f) fully activated integrin αIIbβ₃ binding moiety, and (g) phosphatidylserine binding moiety; (iii) quantifying binding of the plurality of sensor agents to the in vitro thrombus determining each sensor agent result; and (iv) generating a thrombolytic profile of the subject by characterizing the binding of each sensor agent as either increased, no change, or decreased as compared to a reference, forming a thrombolytic profile of a subject comprising binding results for each sensor agent in an ordered readout. In certain aspects the conformation independent integrin αIIbβ₃ binding moiety is a SZ22 antibody; the fibrinogen (Fg) binding moiety is fibrinogen; Von Willebrand Factor (VWF) binding moiety is a 2.2.9 antibody; the P-selectin binding moiety is a AK4 antibody; the conformationally extended integrin βubβ₃ binding moiety is a MBC 370.2 antibody; the fully activated integrin αIIbβ₃ binding moiety is a PAC-1 antibody, and the phosphatidylserine binding moiety is Annexin V.

Other embodiments are directed to a kit for performing a thrombus profile assay as described herein. The kit can include (a) a conformation independent integrin αIIbβ₃ binding moiety, (b) fibrinogen (Fg) binding moiety, (c) Von Willebrand Factor (VWF) binding moiety, (d) P-selecting binding moiety, (e) conformationally extended integrin αIIbβ₃ binding moiety, (f) fully activated integrin αIIbβ₃ binding moiety, and (g) phosphatidylserine binding moiety. The kit can include conjugation reagents for labeling the binding moieties. The kit can include a microfluidic device configured for inducing shear stress thrombus formation.

As used herein a thrombus profile or sensor agent profile refers to profile generated using a blood sample.

As used herein an effect profile or effective profile is a comparative profile the indicates a possible effect of a treatment or agent.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1J. Combining microfluidic stenosis assay with multi-fluorescence imaging to comprehensively characterize biomechanical platelet aggregation. (A) A microfluidic chip with a quarter coin placed adjacently. (B) Illustration of experimental setup. (C) Zoom-in of the dashed box in (B). A hump inside the channel creates 80% stenosis. When blood is perfused over, platelets spontaneously aggregate around the hump. (D) Shear rate and shear stress at the stenosis area estimated by fluid dynamics simulation. (E) Left: a layout of the two sets of fluorescently tagged sensors for thrombus profiling. Right: zoom-in of the thrombus shown in (C), illustrating the staining of Sensor Set 1. (F) Representative fluorescent images of thrombi stained with Sensor Set 1 (left) and 2 (right). (G) Representative time courses of signal intensity of biomarkers in Sensor Set 1 (left, detecting platelets (Plt), fibrinogen (Fg), VWF and P-selectin) and 2 (right, detecting platelets, phosphatidylserine (PS), extended integrin αIIbβ₃ (E⁺αIIbβ₃) and fully activated αIIbβ₃ (Act. αIIbβ₃)). AFU: arbitrary fluorescence unit. (H,I) Scatter plots with mean±s.e.m. (n=28) of the signal intensity of all biomarkers (H; expired and refrigerated blood samples were tested as controls, n=4) and the normalized signal intensity of Fg, VWF, P-selectin, PS, E⁺αIIbβ₃ and Act. αIIbβ₃ (i) 7.5 min after the onset of thrombus formation. The definition of each dimension of the 7-dimension thrombus profile is indicated below the graphs. (J) Scatter plots with mean±s.e.m. (n>3) of the thrombus residue size in the presence of aspirin (2x) or clopidogrel (2x) or both (2x or 20x), or ALB cocktail. N.S., not significant, compared with no drug treatment, assessed by one-way ANOVA (F-value=2.87, degrees of freedom=21) and multiple comparison (p=0.9848, >0.9999, >0.9999, >0.9999, =0.0447, respectively, from left to right).

FIG. 2A-2M. Delineating the respective contribution of GPIba-VWF, αIIbβ₃-VWF and αIIbβ₃-fibrinogen interactions to biomechanical platelet aggregation. (A) (Left) Illustration of key receptor-ligand interactions in a biomechanical thrombus, highlighting the GPIba-integrin αIIbβ₃mechanosensing axis. A panel of monoclonal antibodies and their respective targets are indicated, which were used to inhibit one receptor or ligand at a time. The head of integrin αIIbβ₃colored green or red respectively denotes the integrin being unrecognizable or recognizable by PAC-1. (Right) Table layout of tested antibodies and their respective antigens and targeting receptor-ligand interactions. (B) Representative images of DiOC₆ (3)-labeled thrombi formed in the presence of different concentrations of NMC4. (C-E) Dose-dependency curves of antibodies against GPIba-VWF (c), αIIbβ₃-VWF (D) and αIIbβ₃-fibrinogen (E) interactions in reducing the size of biomechanical thrombi (mean±s.e.m.). (F-L) Comparing the normalized signal intensities of Fg, VWF, P-selectin, PS, E⁺αIIbβ₃and Act. αIIbβ₃ in the biomechanical thrombi, in the absence and presence of AK2 (F), NMC4 (G), LJ-P5 (H), 152B6 (I), 7E9 (J), LJ-155B39 (K) and LJ-134B29 (L), respectively (mean±s.e.m.; n=5). P-values are results of multiple t-test with points without and with drug treatment paired. (M) Summarizing the effects of AK2, NMC4, LJ-P5, 152B6, 7E9, LJ-155B39 and LJ-134B29 on the thrombus profile into 7-digit barcodes. A positive, neutral or negative effect is denoted by a bar being at the top, middle and bottom of the column, respectively; it is also numerically denoted by ‘+’, ‘0’ or ‘−’, respectively. The antibodies are categorized by their target receptor-ligand interaction, which is indicated by different background colors.

FIG. 3A-3F. Testing the effects of inhibitors against integrin αIIbβ₃, VWF and soluble agonists on biomechanical platelet aggregation. (A) Dose dependency of 7E3 and 10E5, in reducing the size of the biomechanical thrombi (mean±s.e.m.). (B) Dose dependency of two sizes (50 and 510 nm) of polystyrene negatively charged nanoparticles (PS-CNP), in reducing the size of the biomechanical thrombi (mean±s.e.m.). (C-E) Comparing the normalized signal intensities of Fg, VWF, P-selectin, PS, E⁺αIIbβ₃ and Act. αIIbβ₃in the biomechanical thrombi, in the absence and presence of 7E3 (C), 10E5 (D) and PS-CNP (diameter: 510 nm) (E), respectively (mean±s.e.m.) (n=4 or 5). P-values are results of multiple t-test with points without and with drug treatment paired. (F) Summarizing the 7-digit effect barcodes of 7E3, 10E5 and PS-CNP. A rule of addition is indicated, demonstrating that the add-up of the barcodes of GPIba-VWF inhibition and αIIbβ₃-VWF inhibition equals that of VWF inhibition, and the add-up of the barcodes of αIIbβ₃-VWF inhibition and αIIbβ₃-Fg inhibition equals that of integrin αIIbβ₃ inhibition.

FIG. 4A-4L. Characterizing abnormalities in the thrombus profiles associated with hypertension and aging. (A) Scatter plots with mean±s.e.m. (n indicated above each column) of the size of thrombi generated by healthy subjects grouped by age. P-values are results of one-way ANOVA (F-value=14.11, degrees of freedom=50) and multiple comparison. (B) Comparing the time course of thrombus growth (mean±s.e.m. with fitting lines of sigmoidal model) between healthy young and hypertensive groups (n=8). (C) Scatter plots with mean±s.e.m. of the thrombus profiles of healthy young, healthy older hypertensive young, and hypertensive older adult subjects (n=33, 14, 9 and 13, respectively). P-values are results of two-way ANOVA (F-values=7.85, 106.3, 43.66; degrees of freedom=18, 6, 3, for interaction, row factor and column factor, respectively) and multiple comparison. (D) P-values of the significance of the impact of aging on the thrombus profile of hypertensive subjects (top), and that of hypertension on the thrombus profile of older subjects. (E) Scatter plot of normalized E⁺αIIbβ₃ signal intensity vs. thrombus size, with blood from healthy young, healthy older, hypertensive young, and hypertensive older subjects (n=33, 14, 9, 13, respectively). Solid line: linear fitting of all data points, with two-sided regression slope test performed to show a significant positive correlation. Dash lines: threshold values that best separate healthy young and other groups. (F) Scatter plots and linear fits (P-values: results of two-sided regression slope test) of thrombus size and normalized E⁺αIIbβ₃ signal intensity vs. hypertension duration, systolic and diastolic blood pressures and their sum, HbAlC, BMI, total cholesterol, LDL-C, HDL-C and triglyceride in hypertension patients. Green, yellow, and red background colors indicate normal, borderline abnormal and pathologically abnormal ranges, respectively. (G-L) Scatter plots with mean±s.e.m. (n indicated above each column) of the thrombus size (left) and normalized E⁺αIIbβ₃ signal intensity (right) of healthy young (G-I) or hypertensive and/or older (j-1) subjects, grouped by gender (G,J), race (H,K) and ethnicity (I,L). One-way ANOVA and multiple comparison or Student's t-test was performed for data comparison, with p-values, F- or t-values and degrees of freedom (df) annotated on the figures.

FIG. 5A-5T. Hyperactivity of GPIba and integrin αIIbβ₃ associated with hypertension. (A) Snapshots of healthy young and hypertensive young subjects' platelets adhering to VWFA1 (upper) or Fg (lower) in flow chamber. (B-D) Mean±s.e.m. (n>3) of surface coverage (B,D) and rolling velocity (C) vs. shear rate of platelets perfused over a surface pre-coated with 25 μg mL-1 VWFA1 (B,C) or 100 μg mL-1 Fg for 1 h (D). P-values are results of two-way ANOVA (F-values=0.183, 13.16, 42.39 (B), 0.352, 3.345, 69.22 (C), 0.768, 6.518, 27.65 (D); degrees of freedom=15, 5, 3 (B), 15, 5, 3 (C), 15, 5, 3 (D) for interaction, row factor and column factor, respectively) and multiple comparison. (E) BFP setup photomicrograph (top) and molecular binding illustration (bottom). (F-K) Adhesion frequency (Scatter plots with mean±s.e.m.) (F,I), effective avidity and affinity (Mean±s.e.m.) (G,J) and bond lifetime vs. force (mean±s.e.m., n≥300 for each curve) (H,K) of VWFA1-(F-H) or Fg-(I-K) coated beads binding to healthy young (HY) or hypertensive (HTN) subjects' platelets. Student's t-test was performed for data comparison, with p-values, t-values and degrees of freedom (df) annotated on the figures. (L) Illustration of fBFP setup. VWFA1 pulling on GPIba triggers intraplatelet Ca²⁺ flux. (M) Representative time course of a hypertensive subjects' platelet's normalized Ca²⁺ level during repeated VWFA1 pulling at 40-pN clamping force. Peak increase A/max is marked. (N) Intraplatelet Ca²⁺ peak increase (scatter plot with mean±s.e.m.) of healthy young (left) and hypertensive (right) subjects' platelets during VWFA1 pulling at different clamping forces. P-values are results of two-way ANOVA (F-values=5.601, 6.146, 63.34; degrees of freedom=3, 3, 1, for interaction, row factor and column factor, respectively) and multiple comparison. F,I,N: different symbol colors indicate data collected from different subjects. (O-R) Representative flow cytometry histograms of E⁺αIIbβ₃(0; n=3), total αIIbβ₃ (P), Act. αIIbβ₃ (Q) and P-selectin (R) signals on healthy young (blue) and hypertensive (red) subjects' platelets. (S) Scatter plot with mean±s.e.m. (n=5) of flow cytometry MFI of total αIIbβ₃, E⁺αIIbβ₃, Act. αIIbβ₃ and P-selectin signals on healthy young (blue) and hypertensive (red) subject's platelets. Multiple t-test was performed for data comparison, with p-values, t ratios and degrees of freedom (df) annotated on the figures. (T) Proposed mechanism model of E⁺αIIbβ₃ over-expression in the biomechanical thrombi of hypertension patients.

FIG. 6A-6E. Drug-disease interactions and personal thrombus barcodes. (A) Individual point plot (n=5; lines connecting points of the same subjects) of the thrombus profiles of hypertension patients without and with aspirin/clopidogrel (2x), NMC4 (IC50) and 7E3 (IC50) treatment. P-values are results of two-way ANOVA (F-values=12.59, 23.13, 34.27; degrees of freedom=18, 6, 3, for interaction, row factor and column factor, respectively). (B) The 7-digit effect barcodes of hypertension without and with NMC4 or 7E3 treatment. A rule of addition is indicated. (C) Illustration of how values in the personal thrombus profiles being low, normal or high are defined. Thrombus profiles of healthy young subjects were used as the reference, the values of which are fitted to a Gaussian distribution. Mean±2s.d. range is defined as normal, and values lower or higher are defined as abnormally low and high, respectively. (D) Fractions of abnormally high, normal and abnormally low values in each dimension of the personal thrombus barcodes from healthy young, healthy older, hypertensive young, hypertensive older, hypertensive+NMC4 and hypertensive+7E3 groups. (E) Comparing the personal thrombus barcodes of hypertensive subjects without and with NMC4 or 7E3 inhibition. Blood samples from a total of 5 subjects was tested. For easier visualization, bars indicating ‘high’, ‘normal’ and ‘low’ are respectively marked by red, yellow, and green.

FIG. 7A-7G. Testing the effect of changing flow perfusion rate on the thrombus profiling results. The perfusion rate was changed from the original 18 μl/min (data acquired from FIG. 4C; n=33 for healthy young and n=9 for hypertensive (HTN) young) to 13.5, 27 and 36 μl/min, respectively. Then total platelet signal intensity (A) and the normalized signal intensity of Fg (B), VWF(C), P-selectin (D), PS (E), E⁺αIIbβ₃ (F) Act. αIIbβ₃(G) were acquired from n=5 healthy young (left, green points) and n=5 HTN young (right, magenta points) subjects and presented as scatter plots with mean±s.e.m. P-values annotated on the graphs are results of two-sided t-tests comparing the result of each HTN young group with that of the healthy young group with the identical perfusion rate. One-way ANOVA was performed to compare the results acquired under different perfusion rates, with the outcome annotated on the graphs (green for healthy young, magenta for HTN young).

FIG. 8. Scatter plot of normalized Fg, VWF, P-selectin, PS, E⁺αIIbβ₃and Act. αIIbβ₃signal intensity vs. total platelet signal intensity, using blood from young healthy subjects and adult subjects with different pathological conditions.

FIG. 9A-9C. Characterizing the effect barcodes of three anti-thrombotic agents using the thrombus profiling assay.

FIG. 10A-10B. An ongoing project exploring partial inhibition of GPIba-VWFA1 interaction to safely inhibit arterial thrombosis. (A) Proposed model. An optimal antithrombotic region may exist that corresponds to a certain range of aggregate residue size in the stenosis assay, in which the thrombotic potential of the blood still remains low while the hemostatic potential already rises to a sufficient level. (B) Dose-dependency curves of three identified partial VWFA1 inhibitors, which at saturating concentrations allow the generation of platelet aggregates with different residue sizes. Hill equation is used to fit the data and derive the bottom (residue size of the thrombus at saturating concentrations of the drug) and IC50.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

Embodiments of the current invention are directed to an effect barcode system that can be used for summarizing the functional effect of different agents/drugs and different pathological conditions. For example, to test an unknown agents function in thrombosis, then it can be introduced it into a blood sample and the evaluation multi fluorescence shear stress evaluation can be performed, and the agent evaluated by analysis of the thrombus profile with and without the agent. Using the condition without the agent as the standard, i.e., with the barcode numerically expressed as [0,0,0,0,0,0,0], the agent is evaluated for results in an increase/decrease in the level of each of the markers. If there's an increase, the corresponding ‘0’ is changed to a ‘+’. If there's a decrease, the corresponding ‘0’ is changed to a ‘−’. If there is no changed the indicator remains ‘0’.

For a pathological condition, the analysis is similar. A subject having a pathological condition (e.g., diabetes, hypertension, aging, smoking, cancer, etc) can be assessed by obtaining a blood sample to run the assay. The averaged thrombus profile can be compared with that of the control population, i.e., healthy young adults. If the pathological condition causes a significant increase in a marker level it as ‘+’ in the effect barcode. If there is a decrease in a marker level it is a ‘−’. If there is no change in marker level is a ‘0’.

### I. Arterial Thrombosis

Arterial thrombosis contributes significantly to global health challenges, with standard diagnostic and therapeutic approaches often proving inadequate and unindicative, especially when confronted with exacerbating factors like hypertension and aging. Overcoming these challenges, a thrombus profiling assay was developed that combines high shear flow and stenotic geometries with multi-color fluorescence imaging to comprehensively characterize thrombi forming under the physiological settings of arterial thrombosis. The assay delineated the complex roles of the multiple platelet receptor-ligand interactions that contribute to biomechanical thrombogenesis. It also uncovered amplified thrombus formation associated with hypertension and aging, indicating a compounded effect of these two risk factors on arterial thrombosis and a paradigm shift in antiplatelet resistance.

The ‘effect barcode’ system of the assay described herein highlights the extended conformation of integrin αIIbβ₃ as a superior marker for detecting platelet hyperreactivity and a potential predictor for arterial thrombosis. By studying drug-disease interactions and characterizing personal thrombus profiles, studies revealed the need of developing different anti-thrombotic therapeutics to specifically accommodate co-existing pathological conditions, and the demand of personalized therapeutic selection (e.g., with the use of effective barcodes), for improving the efficacy and reducing the risk of adverse outcomes in managing thrombotic disorders.

The innovative methodology described herein transcends the experimental setup itself, which also includes the ‘thrombus profile,’ the barcode systems, and the ‘addition rule’ as conceptual elements, thereby establishing a complete platform for evaluating arterial thrombosis. Distinct from traditional coagulation assays (Zhang et al., Frontiers in pharmacology 12, 779753, 2021; Gorog and Becker, Journal of thrombosis and thrombolysis 51, 1-11, 2021), the thrombus profiling assay incorporates high shear flow and stenotic geometries that are physiologically integral to arterial thrombosis. Via testing clinical samples, this assay has demonstrated its translational potential for precisely assessing risks of arterial thrombosis, which could become a game-changer for clinical diagnostics.

The findings reveal that hypertension and aging not only add incremental risks but synergize to amplify thrombotic potential, which provide insights into the mechanistic underpinnings of arterial thrombosis in these populations (Previtali et al., Blood Transfus 9, 120-138, (2011), and also challenge the existing paradigms of antiplatelet resistance observed in these populations (Akturk et al., Clin Appl Thromb Hemost 20, 749-754, 2014; Liu et al., J Geriatr Cardiol 10, 21-27, 2013). The increased activity of GPIba and the intermediate activation state of integrin αIIbβ₃ in hypertensive patients, despite controlled blood pressure, suggest a complex interplay of other pathological factors, e.g., oxidative stress and inflammation (Griendling et al., Circulation research 128, 993-1020, 2021), that warrants further investigation.

The fact that only inhibitors of GPIba-VWF, but not integrin αIIbβ₃-VWF and/or integrin αIIbβ₃-Fg interaction, weakened integrin αIIbβ₃ activation in the thrombus confirmed GPIba mechano-signaling as the primary player in triggering integrin αIIbβ₃ intermediate state activation (Chen et al., Nature materials 18, 760-769, 2019). On the other hand, inhibiting either VWF or Fg binding to integrin αIIbβ₃ did not enhance the recruitment of the other ligand, but both reduced the thrombus size, suggesting that VWF and Fg cooperate, rather than mutually compensate, in integrin αIIbβ₃ crosslinking for biomechanical platelet aggregation. Importantly, the results on these inhibitors appear to indicate that the effective barcode of each anti-thrombotic agent is decided by its target rather than its pharmacological design. Taking advantage of this principle, our experimental system can act as a one-step platform to both screen anti-thrombotic agents and use their effective barcodes to reason backward their therapeutic targets. To serve this purpose, inhibition of other contributing factors of biomechanical platelet aggregation, e.g., intracellular signaling of GPIba and integrin αIIbβ₃ as well as Piezo (Dai et al., Blood 106, 1975-1981, 2005; Shen et al., Nature 503, 131-135, 2013; Zhao et al., Journal of thrombosis and haemostasis: JTH, 2021), shall be tested to acquire their respective effective barcodes.

P-selectin and Act. αIIbβ₃ are widely used markers of platelet activation, but their performance in diagnosing thrombosis is less satisfactory due to low sensitivity (Chung et al., Journal of thrombosis and haemostasis: JTH 5, 918-924, 2007). The inventors have shown that E⁺αIIbβ₃ has a much better performance than P-selectin and Act. αIIbβ₃in correlating with the biomechanical thrombus size and in separating healthy young subjects and subjects carrying prothrombotic risk factors. Furthermore, only E⁺αIIbβ₃, but not P-selectin or Act. αIIbβ₃, was detected on platelets freshly collected from hypertension patients. These results underscore E⁺αIIbβ₃to be more accurate and sensitive than P-selectin and Act. αIIbβ₃in detecting platelet hyperreactivity, which may have the potential to serve as an independent marker for predicting arterial thrombosis.

The vast landscape of anti-thrombotic strategies developed over the past decades evaluated the anti-thrombotic efficacy solely based on thrombus size reduction, and also ignored the influence of co-existing pathological conditions in validation models (Chen and Ju, Stroke Vasc Neurol 5, 185-197, 2020; Gawaz et al., Nature reviews. Cardiology 20, 583-599, 2023). The inventors have demonstrated that thrombi possess multidimensional characteristics that can be orthogonal, which should be summarized as a ‘profile’. Different individuals have sharply different thrombus profiles, manifested by diversified personal thrombus barcodes. Because each antiplatelet agent impacts the thrombus profile in a unique way, it can potentially cause a treatment mismatch in clinical settings if its effect barcode conflicts with the personal thrombus barcode of the patient. This is exemplified by our drug-disease interaction study on hypertension: the suppressed VWF recruitment caused by NMC4 may compromise hemostasis, whereas the persisting high E⁺αIIbβ₃ level after 7E3 inhibition suggests a remaining threat of thrombosis (FIG. 14). The treatment mismatch phenomenon may account for the resistance of certain populations to conventional antiplatelet drugs (Angiolillo et al., Journal of the American College of Cardiology 50, 1541-1547, 2007; Akturk et al., Clin Appl Thromb Hemost 20, 749-754, 2014; Liu et al., J Geriatr Cardiol 10, 21-27, 2013; Jalalian et al., Egypt Heart J 75, 28, 2023), where persisting platelet hyperreactivity causes relapse of thrombosis. By the same token, increased risks of myocardial infarction were indicated with long-term therapy of conventional integrin αIIbβ₃ antagonists that stimulate integrin αIIbβ₃ activation (Chew et al., Circulation 103, 201-206, 2001; Lin et al., Cell 185, 3533-3550 e3527, 2022). Studying the effect barcodes of antiplatelet drugs and their interactions with thrombosis-exacerbating factors can help avoid treatment mismatch, while the inter-individual variations in drug efficacy further urges the application of personalized medicine in treating arterial thrombosis: in order to reach optimal treatment outcomes, drugs with a counteractive effect barcode tailored to the patient's thrombus barcode shall be tested in the patient's own blood to determine the right combination and dosage.

### II. Thrombus Profiling Assay

Embodiments of the invention can include a device to simulate shear stress induced thrombogenesis. Fluidic microchips have been designed to mimic stenosis by providing a microchannel having a stenotic segment. In certain embodiment a microfluidic chip is composed of rectangular channels. In certain aspects the rectangular channels can be 200×50 μm channels in parallel. The inlet of each channel is connected via tubing to a blood sample and the outlet to a syringe or other means of motive force. The syringe can be driven by a pump to perfuse blood through the microfluidic channel at a predetermined flow rate, within which a stenosis site stimulates platelet aggregation towards thrombogenesis. A selected perfusion rate is used to create a sufficient wall shear stress (WSS) at the stenosis site according to fluid dynamics simulation. The same stenosis site WSS can be achieved by an inlet site wall shear rate in a circular vessel with the same cross-sectional area, mimicking human arterioles. The same shear rate is also found in large human arteries during systole and in mouse arteries. To reach the same stenosis site WSS in a square channel requires a low perfusion rate, which can cause steplike movement of the syringe pump and undesired pulsatile flow.

To characterize the thrombus formed inside the stenotic channel, a plurality of biomarkers selected together with their respective molecular sensors can be used. The molecular sensors can include but are not limited to:
(i) Platelets can be reported by SZ22, a monoclonal antibody (mAb) that reacts with a chain of CD41 on platelets and megakaryocytes, but does not inhibit platelet aggregation. SZ22 binds integrin αIIbβ₃ in all conformations, i.e., is a conformation independent antibody (see Chen et al., Nature materials 18, 760-769, 2019, which is incorporated herein by reference). Platelets, or thrombocytes, are small, colorless cell fragments in our blood that form clots and stop or prevent bleeding.
(ii) Fibrinogen (Fg) can be reported by purified human Fg. Exogenous Fg functions as endogenous Fg does, exogenous Fg incorporates into the thrombus like the endogenous Fg. Incorporation of labeled exogenous Fg is an indicator of the relative enrichment of the Fg in the thrombus. Fibrinogen is a soluble protein present in blood plasma, from which fibrin is produced by the action of the enzyme thrombin.
(iii) von Willebrand Factor (e.g., uniprot P04275) can be reported by mAb 2.2.9 (see Fulcher and Zimmerman, PNAS 79:1648-52, 1982) without functional inhibition (see Dent et al., The Journal of clinical investigation 88, 774-782, 1991, which is incorporated herein by reference). VWF, is an adhesive glycoprotein that mediates adhesion and aggregation of platelets at sites of vascular injury.
(iv) P-selectin (e.g., uniprot P16109) can be reported by monoclonal antibody AK4 to signify platelet α-granule release following activation (see Kamath et al., European heart journal 22, 1561-1571, 2001 which is incorporated herein by reference). P-selectin, also known as CD62P, is a member of the selectin family of cell adhesion molecules. It is expressed on stimulated endothelial cells and activated platelets and mediates leukocyte rolling on stimulated endothelial cells and heterotypic aggregation of activated platelets onto leukocytes).
(v) Phosphatidylserine (PS) exposure in the cell membrane can be reported by Annexin V (e.g., uniprot P08758), which signifies the platelets gaining procoagulant function (see Schoenwaelder et al., Blood 114, 663-666, 2009 which is incorporated herein by reference). Phosphatidylserine (PS) is a phospholipid component of the cell membrane. It plays a key role in cell cycle signaling, specifically in relation to apoptosis. Its exposure on the outer surface of a membrane marks the cell for destruction via apoptosis. Annexin V is a cellular protein in the annexin group. Annexin V is commonly used to detect apoptotic cells by its ability to bind to phosphatidylserine, a marker of apoptosis when it is on the outer leaflet of the plasma membrane.
(vi) Conformationally extended (E⁺) integrin αIIbβ₃ can be detected by monoclonal antibody MBC370.2. MBC370. 2 recognizes a motif on the αIIb Calf-1 domain only accessible on extended integrins. Integrin αIIbβ₃, also known as glycoprotein IIb/IIIa (GPIIb/IIIa), is an integrin complex found on platelets. It is a transmembrane receptor for fibrinogen and von Willebrand factor, and aids platelet activation. The complex is formed via calcium-dependent association of gplIb and gpIlla, a required step in normal platelet aggregation and endothelial adherence. Platelet activation by ADP leads to the aforementioned conformational change in platelet gplIb/IIIa receptors that induces binding to fibrinogen.
(vii) Fully activated (Act.) integrin αIIbβ₃ can be detected by monoclonal antibody PAC-1.

The combination of (vi) and (vii) report the integrin activation status (Chen et al., Nature materials 18, 760-769, (2019). The above sensors (detectable affinity agents) can be grouped into 2 or more sets/combinations of 2, 3, 4, 5, or 6 sensors in a set for fluorophore conjugation SZ22 can appear in both sets for reference. These sensors were confirmed to have negligible influence on thrombogenesis.

A thrombus profiling assay successfully observed fluorescence signals from all 7 biomarkers or related sensors. Real-time tracking showed an initial phase of rapid thrombogenesis in the first 300-400 s followed by a second phase of slower development. In one instance the time point for thrombus profiling was selected to be 450 s after the onset of thrombogenesis. Thrombus profiling can be performed at 350, 375, 400, 425, 450, 475, 500, 525, 550 s after the onset of thrombogenesis, including all values and ranges there between. Signal intensities of the sensors can be normalized over that of platelets to assess their enrichment in the thrombus.

Using a thrombus profiling assay described above and taking advantage of a panel of highly specific inhibitory monoclonal antibodies (mAbs), different platelet-crosslinking mechanisms contributing to biomechanical platelet aggregation can be investigated. To succinctly express the effects of different agents to biomechanical platelet aggregation, an “effect barcode” system with 7 columns was created, each column corresponding to one dimension of the 7-dimension thrombus profile. A positive, neutral, or negative effect of an agent to the readout of a dimension is respectively represented by a black bar being at the top, middle or bottom of the column, which can also be numerically expressed as ‘+’, ‘0’ or ‘−’. Using this system, the effects of various reagents or the characterization of a particular sample can be profiled, compared, and/or assessed.

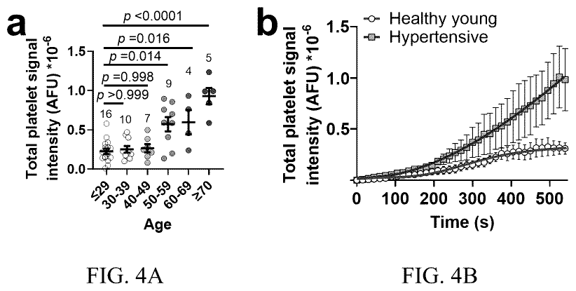

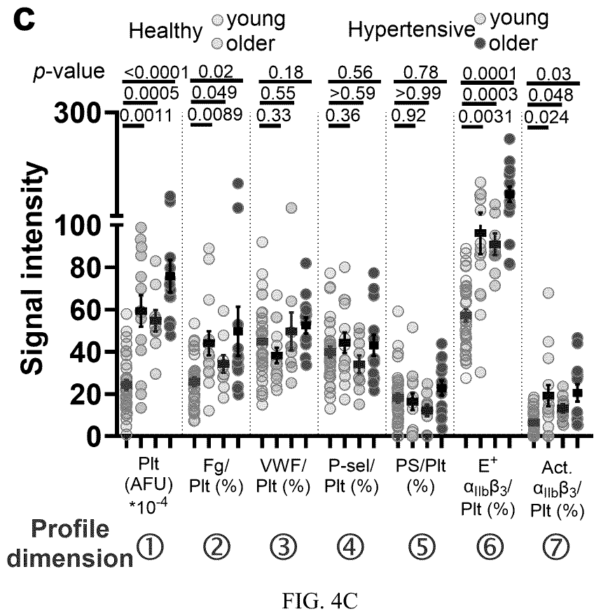

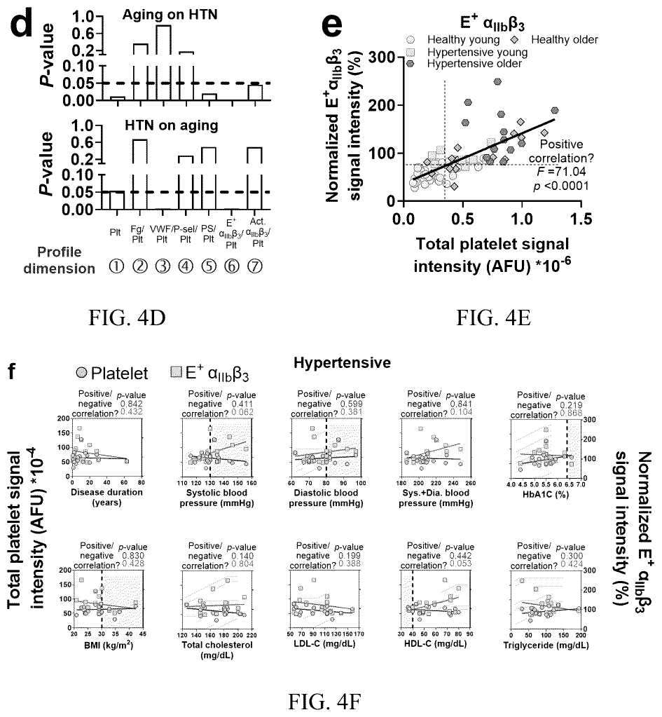

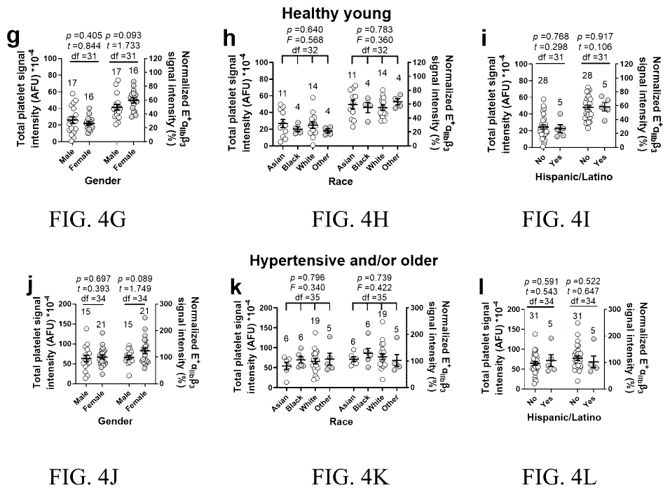

Anti-thrombotic inhibitors effect on the thrombus profile of hypertension patients was tested. At IC50, NMC4 reduced the thrombus size and E⁺and Act. αIIbβ₃ expression to the healthy level, but also reduced VWF enrichment and P-selectin expression that were not up-regulated by hypertension (FIG. 4m). The resulting effect barcode, [0+−−0 0 0], equals the add-up of the effect barcodes of NMC4 and hypertension (FIG. 4n). Similarly, adding 7E3 to hypertension patients' blood resulted in an effect barcode of [0 0−0 0++], which equals the add-up of the effect barcodes of 7E3 and hypertension (FIG. 4m, n). These results indicate that the addition rule of the barcode system also applies to predict drug-disease interactions. Neither NMC4 nor 7E3 corrected the effect barcode of hypertension back to healthy (i.e., [0 0 0 0 0 0 0]), indicating a treatment mismatch between the inhibitors and the patients.

The results described in the Examples section below indicate the translational potential of the thrombus profiling assay for the diagnosis of thrombotic risks and the personalized selection of anti-thrombotic drugs. To evaluate the normality and abnormalities of an individual's thrombus profile, the “effect barcode” or “effective barcode” system can be modified to create personal thrombus barcodes. From healthy young subjects' thrombus profiles, values of each dimension were fitted to a Gaussian distribution. The range within mean±2S.D. of the Gaussian fits (95% confidence interval) was defined as normal (‘0’), and values lower or higher than this range are defined as abnormally low (‘−’) and high (′+′), respectively. All dimensions of healthy young subjects' personal thrombus profiles were dominated by normal values; in contrast, much larger proportions of healthy elderly, hypertensive young, and hypertensive elderly subjects had high values in the thrombus size and E⁺and Act. αIIbβ₃ levels, while the fraction of high Fg level was also moderately higher. These results indicate strong inter-individual variation in the personal thrombus barcode that cannot be completely ascribed to disease and aging, and demonstrate obvious decoupling of the different dimensions in the thrombus profile.

Inter-individual variation was also observed in the efficacy of anti-thrombotic inhibitors. Consistent with the results described in the Examples section, the same dose of NMC4 effectively corrected the thrombus size and the E⁺αIIbβ₃ level in most hypertension patients' blood. However, it did not uniformly modify the personal thrombus barcodes of all 5 patients, but instead produced three different barcodes: [0 0 0 0 0 0 0], [0 0-00 0 0] and [0 00 0 0+0].

### A. Microfluidic Stenosis Assay.

Microfluidic devices with single-hump stenosis microchannels can be coated with VWF monomer solution. Heparin added whole blood is incubated with DiOC₆ (3), or with a set of predetermined sensors. As used herein, a sensor is a compound or protein (e.g., antibody) that specifically binds a selected molecule, peptide, or protein present in a thrombus, the sensor can optionally include a detectable tag. A detectable tag can include but is not limited to a fluorescent tag. In certain aspects a first sensor set (Sensor Set 1) can include one or more of SZ22-FITC, Fg-Alexa Fluor® 405, 2.2.9-Alexa Fluor® 555 and AK4-Alexa Fluor® 647. In certain aspects a second sensor set (Sensor Set 2) can include SZ22-FITC, Annexin V-Pacific Blue, MBC 370.2-Alexa Fluor® 555 and PAC-1-Alexa Fluor® 647. A thrombogenic sample, e.g., blood, can be introduced via a reservoir at a microchannel inlet and perfused through the microchannel at a predetermined flow rate. In certain aspects the sample is moved through the microchannel using a pump withdraw mode via a syringe or similar apparatus connected to a microchannel outlet. The microchannel at the site of stenosis can be recorded in real time with multi-fluorescence imaging using appropriate equipment. In certain aspects the multi-fluorescence imaging is performed with a LED5 fluorescence light source and a multi-pass filter (excitation-emission: 391/32-435/30, 479/33-519/25, 554/24-594/32 and 638/31-695/58 nm). Autofluorescence of platelets can be detected in the 391/32-435/30 nm channel (Lohmann and Lohmann, Biochemical and biophysical research communications 152, 1410-1415, 1988), the signal intensity of which can be subtracted when calculating signals from other sensors.

### B. Blood Collection, Reconstitution and Platelet Isolation.

For whole blood stenosis assay, blood can be slowly drawn from the vein of a volunteer into a syringe pre-loaded with heparin. For laminar flow chamber assay, blood can be drawn into a syringe pre-loaded with ACD buffer at a 5:1 ratio. Platelet counting is performed, and the blood is centrifuged at 200 g. A fraction of the platelet rich plasma is removed to reduce the final platelet count. Modified Tyrode buffer pH 6.5 is added to reach the original blood volume, and prostaglandin E1 is added to prevent platelet activation. The blood is centrifuged at 1600 g. The supernatant is removed and MTB pH 6.5 and prostaglandin E1 were added again, together with apyrase. The blood is centrifuged again at 1600 g. The supernatant is removed, and MTB pH 7.4 with 50 mg mL-1 BSA is added to the blood to reach a final hematocrit of 45%. Finally, the reconstituted blood was added with prostaglandin E1 and apyrase.

For platelet isolation, blood is drawn to a syringe preloaded with ACD buffer at a 5:1 ratio, transferred into a 15 ml tube pre-loaded with apyrase (e.g., 0.005 U per ml blood) and clexane (e.g., 20 U per ml blood), and centrifuged at 200 g without brake. Platelet-rich plasma is extracted and centrifuged at 600 g. The platelet pellet is resuspended into platelet washing buffer (e.g., 4.3 mM K₂HPO₄, 4.3 mM Na₂HPO₄, 24.3 mM NaH₂PO₄, 113 mM NaCl, 5.5 mM D-Glucose, 10 mM theophylline and 1% BSA, pH 6.5) pre-added with Clexane and apyrase, and centrifuged at 600 g. The platelet pellet is resuspended into Hepes-Tyrode buffer (134 mM NaCl, 12 mM NaHCO₃, 2.9 mM KCl, 0.34 mM sodium phosphate monobasic, 5 mM HEPES, and 5 mM glucose, 1% BSA, pH 7.4) pre-added with apyrase.

### C. Microfluidic Device Preparation.

In certain embodiments a microfluidic device can be prepared. For example, an SU-8 mold can be fabricated on a silicon wafer with 1-μm resolution. Polydimethylsiloxane (PDMS) is applied on the mold, which were together heated for curing. The cured PDMS can be peeled off the mold and cut into single devices. Holes are drilled on the devices to create microchannel outlets and microchannel inlets. The devices then undergo plasma treatment and are bonded to glass coverslips, which are heated to achieve firm attachment.

### III. Statistical analysis

Comparison between two groups were conducted by two-tailed Student's t-test or multiple t-test. Comparison between three or more groups were conducted by one-way or two-way analysis of variance (ANOVA). Ratio paired t-test was used to compare the sizes of thrombi generated using the same blood sample stained respectively with Sensor Sets 1 and 2. Regression slope test was used to assess whether the slope of a linear regression is significantly non-zero.

To compare thrombus profiling results from healthy young, healthy elderly, hypertensive young and hypertensive elderly groups, two-way ANOVA with unequal sizes and heteroscedastic factorial designs was used (Yates, Journal of the American Statistical Association 29, 51-66, 1934). A Box-type finite-sample approximation method was applied for the distribution of quadratic forms (Brunner et al., Journal of the American Statistical Association 92, 1494-1502, 1997). Specifically, we denote the size of each group as nᵢⱼ (i=1 (young) or 2 (elderly) and j=1 (healthy) or 2 (hypertensive)). Assume that the data points in each group are independent and follow the normal distribution N (μᵢⱼ, σᵢⱼ²) (i=1,2, j=1,2), where (μ₁₁/μ₁₂/μ₂₁, μ₂₂) are the respective means of different groups and (σ₁₁², σ₁₂², σ₂₁², σ₂₂²) are the respective variances of different groups. According to the model assumption of a two-way ANOVA:

$${μ}_{ij} = {μ}_{0} + {α}_{i} + {β}_{j} + {γ}_{i j} ( i = 1 , 2 , j = 1 , 2 ) \qquad ( 4 )$$

where μₒ is the common mean, αᵢ denotes the main effect of the hypertension factor, βⱼ denotes the main effect of the aging factor, and γᵢⱼ denotes the interaction effect from both factors. The null hypotheses for two main effects and one interaction effect are as follows:

$${H}_{01} : {α}_{1} = {α}_{2} = 0$$

$${H}_{02} : {β}_{1} = {β}_{2} = 0$$

$${H}_{03} : {γ}_{11} = {γ}_{12} = {γ}_{21} = {γ}_{22} = 0$$

For each effect, we need to calculate the quadratic form statistics (Brunner et al., Journal of the American Statistical Association 92, 1494-1502, 1997). Let I be a 2× 2 identity matrix and J a 2×2 matrix of 1's. The symbol ⊗ denotes the Kronecker product of two matrices. We define

$${M}_{1} = P ⊗ \frac{1}{2} J , {M}_{2} = \frac{1}{2} J ⊗ P , and {M}_{3} = P ⊗ P , where P = I - \frac{1}{2} J .$$

Then, the above three null hypotheses of interest can be written using the unified form of

$${H}_{0 k} : {{M}_{k} ( {μ}_{11} , {μ}_{12} , {μ}_{21} , {μ}_{22} )}^{′} = 0$$

for k=1,2,3. Define the sample mean and sample covariance of data in each group as X̅ and Ŝ, and define matrix Dₖ=diag(Mₖ). Then the quadratic form test statistics for the k-th effect is defined as

$${F}_{k} = \frac{N \cdot {\overline{X}}^{′} {M}_{k} \overline{X}}{tr ( {D}_{k} \hat{S} )}$$

for k=1,2,3. According to the Box-type approximation (Box, The Annals of Mathematical Statistics, 290-302, 1954), the distribution of Fₖ can be approximated by a F distribution F_{(f*}_{ₖ}_{,f*}_{ₒₖ}₎, where

$${\hat{f}}_{0 k} = \frac{{( tr ( {D}_{k} \hat{S} ) )}^{2}}{tr ( {D}_{k}^{2} {\hat{S}}^{2} Λ )} and {\hat{f}}_{k} = \frac{{( tr ( {D}_{k} \hat{S} ) )}^{2}}{tr ( {M}_{k} \hat{S} {M}_{k} \hat{S} )} ,$$

in which

$$Λ = diag \{ \frac{1}{{n}_{11} - 1} , \frac{1}{{n}_{12} - 1} , \frac{1}{{n}_{21} - 1} , \frac{1}{{n}_{22} - 1} \} .$$

Using the above equations, we obtained a p-value corresponding to each dimension of the thrombus profile from the approximated distribution.

### IV. Biomarkers

A biomarker is an organic biomolecule that is differentially present in a sample from a subject of one phenotypic status (e.g., having a disease or condition) as compared with another phenotypic status (e.g., not having the disease or condition). Biomarkers, alone or in combination, provide measures of relative risk that a subject belongs to one phenotypic status or another. As such, they are useful as markers for disease (diagnostics), therapeutic effectiveness of a drug (theranostics) and of drug toxicity.

Proteins and antibodies. Human fibrinogen Integrin αIIbβ₃-targeting antibodies SZ22 and P2, AK4, PAC-1, AK2, HIP-8, MBC 370.2, 2.2.9, NMC4, LJ-P5, 152B6, LJ-155B39, LJ-134B29 and recombinant human VWFA1 (residues A0742-17/21) (Ruggeri et al., Blood 108, 1903-1910, 2006) monomer were from MERU VasImmune (San Diego, CA, USA). VWF monomer was from Sino Biological (Wayne, PA, USA). 7E9, 7E3 and 10E5 were generous gifts from Dr. Barry S. Coller (The Rockefeller University, New York, NY, USA).

As used herein, the term “antigen” or “sensor target” is a molecule capable of being bound by an affinity agent (e.g., antibody). The structural aspect of an antigen, e.g., three-dimensional conformation or modification (e.g., phosphorylation), giving rise to a biological response is referred to herein as an “antigenic determinant” or “epitope.” Thus, antigenic determinants or epitopes are those parts of an antigen that are recognized by antibodies. An antigenic determinant need not be a contiguous sequence or segment of protein and may include various sequences that are not immediately adjacent to one another. In certain embodiments, binding moieties other than antibodies can be used to specifically bind to an antigen, e.g., non-antibody proteins, aptamers, avimers, and the like.

The term “antibody” or “immunoglobulin” is used to include intact antibodies and binding fragments/segments thereof. Typically, fragments compete with the intact antibody from which they were derived for specific binding to an antigen. Fragments include separate heavy chains, light chains, Fab, Fab′ F(ab′) 2, Fabc, and Fv. Fragments/segments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibodies. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin Exp Immunol 79:315-21, 1990; Kostelny et al., J. Immunol. 148:1547-53, 1992.

The phrase “specifically binds” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody to be selected for its specificity to the target.

Labeling. Affinity agents of the invention can be conjugated with various detectable label to measure the abundance of a thrombus component. A detectable label or sensor incorporating a detectable label can include, but is not limited to FITC, Alexa Fluor® 488, Alexa Fluor® 647, Alexa Fluor® 647, Alexa Fluor 488, Annexin V-Pacific Blue, Annexin V-Alexa Fluor® 488, heparin, Alexa Fluor® 405, Alexa Fluor®555, Alexa Fluor® 647, PS-CNP beads, and DiOC₆ (3). DiOC₆ (3) (3,3′-Dihexyloxacarbocyanine Iodide) is a cell-permeant, green-fluorescent, lipophilic dye that is selective for the mitochondria of live cells, when used at low concentrations. At higher concentrations, the dye may be used to stain other internal membranes, such as the endoplasmic reticulum.

The affinity agents of the present invention are used for fluorescent assays and can be detectably labeled with fluorophores. There are a wide variety of fluorophore labels that can be attached to the affinity agents of the present invention. Common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCPCy5.5, PE-Cy5, PE-Cy5.5, PE-Cy7, PE-Texas Red, and APC-Cy7. Other fluorophores include, inter alia, Alexa Fluor® 350, Alexa Fluor® 488, Alexa 25 Fluor® 532, Alexa Fluor® 546, Alexa Fluor® 568, Alexa Fluor® 594, Alexa Fluor® 647 (monoclonal antibody labeling kits available from Molecular Probes, Inc., Eugene, OR, USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, OR, USA), and Cy2, Cy3, Cy3.5, Cy5, Cy5.5, Cy7, all of which are also useful for fluorescently labeling the affinity agents of the present invention. For secondary detection using labeled avidin, streptavidin, captavidin or neutravidin, the affinity agents of the present invention can usefully be labeled with biotin.

### V. Anti-Thrombotic Therapy

Thrombosis is the formation of a thrombus (blood clot) in a blood vessel or an organ (a chamber of the heart). Clots can block blood flow in your blood vessels or break free and travel elsewhere in the body. A clot can disrupt blood flow to an organ and result in a life-threatening emergency. Symptoms vary based on the clot's location and can include chest pain, trouble breathing and skin changes. Some people face a higher risk of thrombosis due to medical conditions or other factors.

Thrombosis treatments include medications, minimally invasive procedures and surgeries. Examples of treatments for thrombosis include: (i) Blood-thinners. These medications keep your blood from clotting too easily. There are two classes of blood-thinners: antiplatelet drugs and anticoagulants. (ii) Thrombolytic therapy. Thrombolytic therapy uses medications to dissolve blood clots. They serve as emergency treatment for heart attacks, strokes and other thrombosis complications. (iii) Thrombectomy. One of the most direct ways to remove a clot is for a surgeon to access it and remove it.

Methods of the invention are useful in assessing a thrombus profile providing information for prevention and treatment of pathologic conditions associated with an increased risk of thrombosis including, but in no way limited to, pulmonary embolism, myocardial infarction, stroke and iatrogenic or spontaneous thrombosis. Increased understanding of the mechanisms underlying thrombosis and of interventions has led to a polypharmacological anti-thrombotic approach utilizing appropriate combinations of anti-platelet, anti-coagulant, and fibrinolytic agents. Examples of anti-thrombotic compounds used include anti-platelet agents such as aspirin, clopidogrel, ticlopidine, GPIIb/IIIa antagonists; anti-coagulants such as thrombin inhibitors, warfarin, heparin and low molecular weight heparins; and fibrinolytic agents including but not limited to, streptokinase, tissue plasminogen activator (tPA) and tenecteplase.

### VI. Kits

In another aspect, the present invention provides kits for performing thrombus profiling assays, which kits are used to characterize and assess in vitro thrombus composition and character as described herein. In one embodiment, the kit comprises a microfluidic device, such as a chip, configured to produce a shear stress induce thrombus. The kit can include affinity reagents and affinity reagent labels. Thus, for example, the kits of the present invention can comprise antibodies conjugated to fluorescent labels.

The kit can also comprise a washing solution or instructions for making a washing solution, in which the combination of the affinity reagent and the washing solution allows evaluation of the biomarker or biomarkers in the in vitro thrombus, e.g., multi fluorescent intensity.

In a further embodiment, such a kit can comprise instructions for suitable operational parameters in the form of a label or separate insert. For example, the instructions may inform a consumer about how to collect the sample, how to manipulate the sample, and/or how to detect/evaluate the particular biomarkers.

In yet another embodiment, the kit can comprise one or more containers with biomarkers or biomarker affinity agents to be used as standard(s) for calibration.

### VII. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Multi-Parametric Thrombus Profiling Microfluidics Uncovers Mechanobiology of Hypertension and Aging-Related Thrombosis

### A. Results

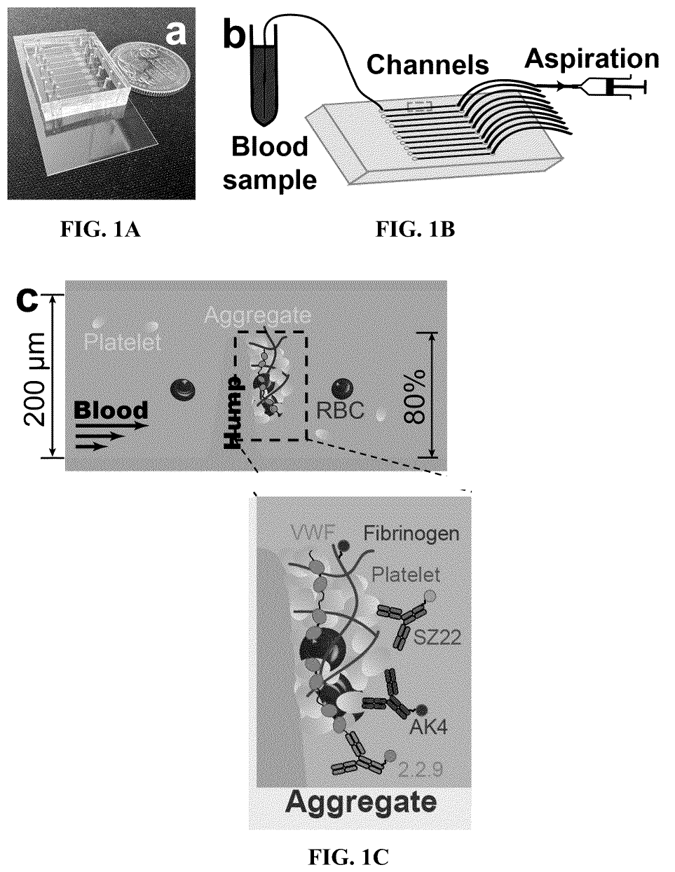

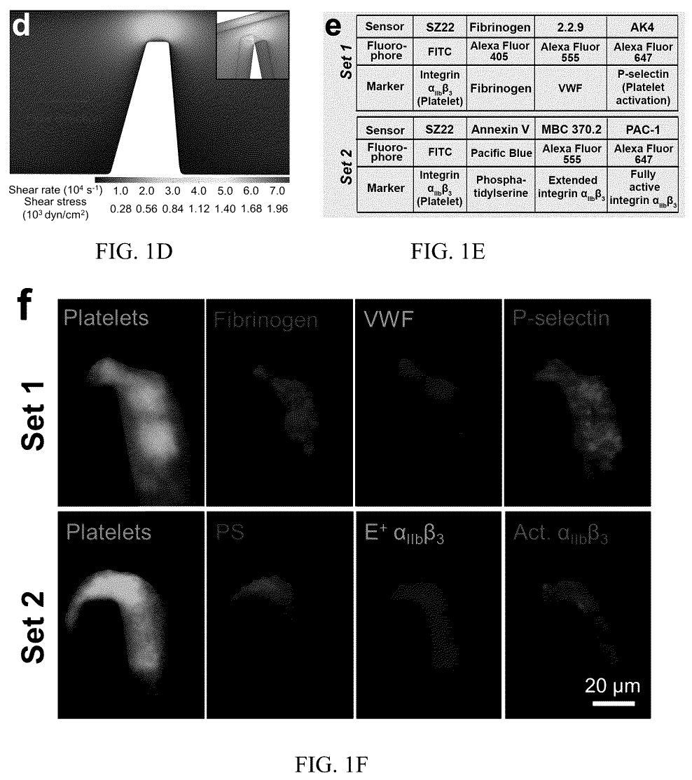

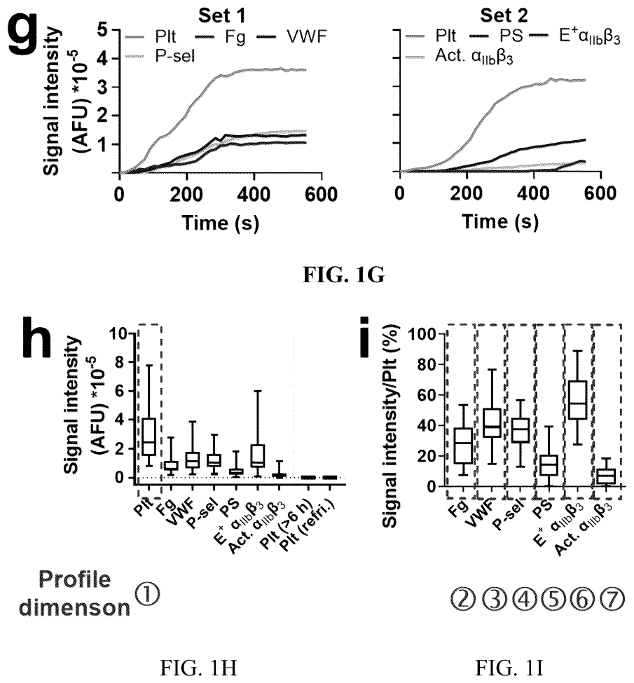

Thrombus profiling assay: development and validation. Each microfluidic chip is composed of 10 rectangular (width×height: 200 μm×50 μm) channels with respective inlets and outlets for tubing connection (FIG. 1A, 1B). A pump drives a syringe to perfuse heparinized blood (0.5 mL) through the channel pre-coated with VWF. An 80% stenosis site stimulates biomechanical thrombogenesis (FIG. 1C). A perfusion rate of 18 μL min⁻¹ was selected, which creates a wall shear stress (WSS) of 857 dyn cm⁻² at the stenosis site according to fluid dynamics simulation (FIG. 1D). The same stenosis site WSS is achieved by an inlet wall shear rate of 1485 s-1 in a circular vessel with the same cross-sectional area, mimicking human arterioles (Yakusheva et al. Blood advances 6, 4834-46), human arteries during systole, and mouse arteries (Panteleev et al., Journal of thrombosis and haemostasis JTH 19, 588-95). The calculated Reynolds numbers in channels of different shapes are also within the same scale. With the above settings, platelet thrombi can be consistently observed within the channel, which is primarily driven by shear force because no external agonist is added to the blood and the high-speed perfusion prevents the localized accumulation of agonists released from attached platelets and red blood cells. Due to the high shear force, most thrombi have a tendency of growing towards the downstream side of the stenosis. Nonetheless, most thrombi (>85%) cover the whole stenosis apex, and most (>85%) thrombi have the point in their contour most close to the opposing channel wall positioned above the stenosis apex, making the stenosis apex still the most vulnerable position for occlusion. Replacing VWF with collagen for channel coating did not significantly affect thrombus formation. However, with collagen coating, thrombus formation was basically eliminated by RU5 which blocks plasma VWF binding to collagen, reflecting an indispensable role of VWF on the hump for platelet attachment. Replacing heparin with citrate or ethylenediaminetetraacetic acid (EDTA) for anticoagulation attenuated thrombus formation, because the latter two chelate calcium from the blood and inhibit platelet activation, while EDTA also eliminates integrin αIIbβ₃ activity. These results validate the use of VWF and heparin for channel coating and blood anticoagulation, respectively.

To comprehensively characterize the thrombus formed inside the stenotic channel, 7 biomarkers were carefully selected together with their respective molecular sensors (FIG. 1E). Platelets are reported by SZ22, a monoclonal antibody (mAb) that stains integrin αIIbβ₃ in all conformations (Chen et al., Nature materials 18, 760-769, 2019). Fibrinogen (Fg) is reported by purified human Fg that spikes the blood at 1% of plasma concentration. Von Willebrand Factor (VWF) is reported by mAb 2.2.9 without functional inhibition (Dent et al., The Journal of clinical investigation 88, 774-782, 1991). P-selectin is reported by AK4 to signify platelet α-granule release following activation (Kamath et al., European heart journal 22, 1561-1571, 2001). Phosphatidylserine (PS) exposure in the cell membrane is reported by Annexin V, which signifies the platelets gaining procoagulant function (Schoenwaelder et al., Blood 114, 663-666, 2009). Conformationally extended (E⁺) and fully activated (Act.) integrin αIIbβ₃ are respectively detected by mAbs MBC370.2 and PAC-1, which in combination precisely report the integrin activation status (Chen et al., Nature materials 18, 760-769, 2019). The above sensors were grouped into 2 sets for fluorophore conjugation (FIG. 1E), where SZ22 appears in both sets for reference. All sensors have negligible influence on thrombogenesis.

Fluorescent signals were observed from all 7 biomarkers (FIG. 1F). Agreeing with previous observations, real-time tracking showed rapid thrombogenesis in the first 300-400 s followed by a quasi-steady phase, in which the thrombus reaches a relative equilibrium between platelet aggregation and disaggregation (Nesbitt et al., Nature medicine 15, 665-73; Brazilek et al., Lab on a chip 17, 2595-608) (FIG. 1G). Thus, we selected 450 s after the onset as the time point for quantitating fluorescent signals so as to assess the thrombus in the fully developed status while avoiding unnecessary waiting (FIG. 1H). Signal intensities of Fg, VWF, P-selectin, PS and E⁺and Act. αIIbβ₃ were normalized by platelet signal to assess their enrichment (FIG. 1I), where the high E⁺αIIbβ₃ signal and low Act. αIIbβ₃ signal agree with our previous discovery that biomechanical platelet aggregation is mainly mediated by an intermediate activation state of αIIbβ₃ integrins (Chen et al., Nature materials 18, 760-69). P-selectin expression and low-level PS exposure observed here (FIG. 1F,I; further confirmed using different microscope setup, staining agents and microfluidic channel design) should be induced by GPIba and/or integrin αIIbβ₃ mechanosignaling (Merten et al., Circulation 102, 2045-50, 2000; Deng et al., Nature communications 7, 12863, 2016; Hu et al., Thromb Haemost 90, 679-87, 2003; Roka-Moiia et al., Thromb Haemost 120, 776-92, 2020; Pang et al., Blood 132, 533-43). The total signal intensity of platelets (1^{st} dimension, indicating thrombus size) and the normalized signal intensities of Fg, VWF, P-selectin, PS and E⁺and Act. αIIbβ₃ (2^{nd}-7^{th} dimensions) are summarized into a 7-dimension thrombus profile (FIG. 1F,I).

Blood stored for >6 h or refrigerated overnight both failed to generate visible thrombi (FIG. 1H), which is likely due to a loss of platelet activity during room temperature storage and GPIba shedding during cold storage (Chen et al., Arteriosclerosis, thrombosis, and vascular biology 36, 1821-28, 2016), respectively. Activated platelets can release and/or help produce soluble agonists such as thromboxane A2, ADP, and thrombin to further activate themselves and recruit surrounding platelets to the growing thrombus, wherein the activation signaling processes are called amplification loops. However, conventional antiplatelets aspirin (targeting thromboxane A2 (TXA2)) and clopidogrel (targeting P2Y₁₂-ADP interaction) rendered negligible inhibition to the biomechanical thrombogenesis both separately and combined at twice or 20 times of human plasmatic concentrations (Arrebola et al. J Cardiovasc Pharmacol 43, 74-82, 2004) (FIG. 1J). In contrast, both drugs can significantly inhibit ADP or collagen-induced platelet aggregation at much lower concentrations (Arrebola et al. J Cardiovasc Pharmacol 43, 74-82, 2004). Also, a platelet ALB cocktail (including apyrase, MRS2179 and 2-MeSAMP to block ADP, indomethacin to block TXA2, and hirudin to block thrombin, all at saturating concentrations) only reduced the thrombus size by ˜20% (FIG. 1J). These results corroborate the previous observations that inhibiting platelet amplification loops is ineffective in inhibiting biomechanical platelet aggregation (Nesbitt et al., Nature medicine 15, 665-73, 2009; Chen et al., Nature materials 18, 760-69, 2019; Li et al., PloS one 9, e82493, 2014), demonstrating a secondary role of soluble agonists in biomechanical thrombogenesis.

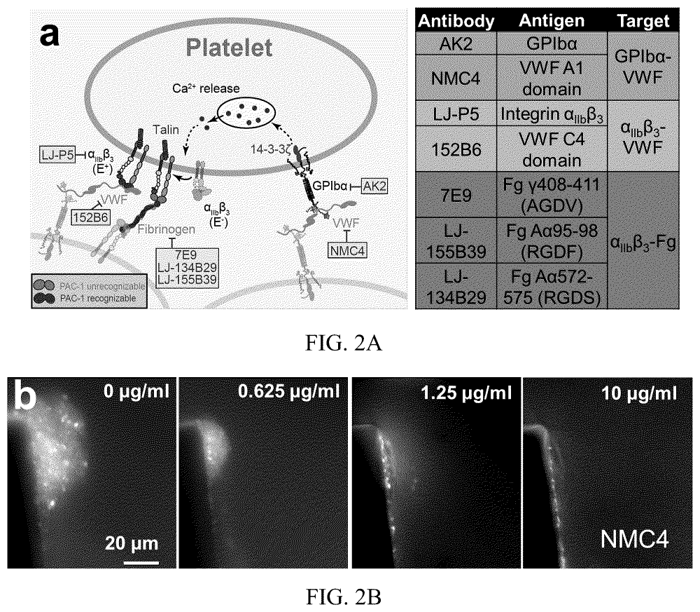

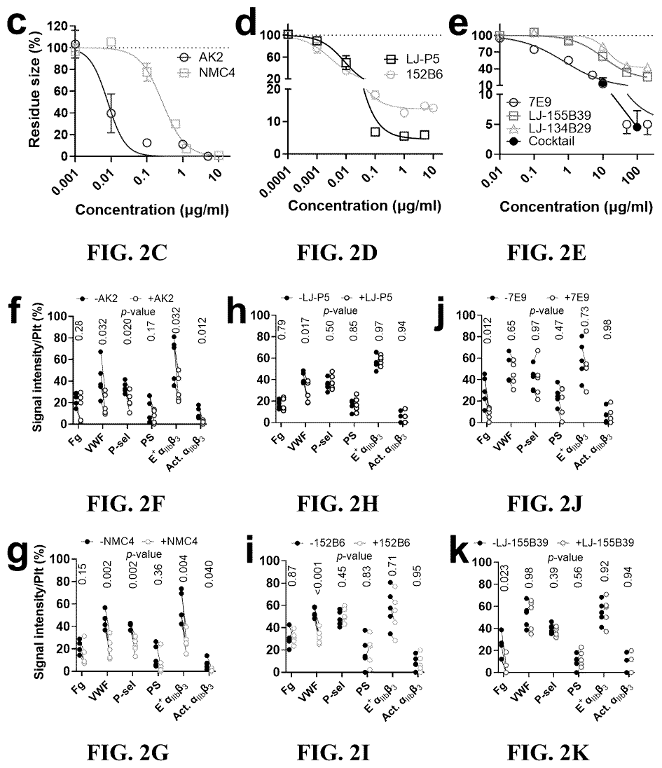

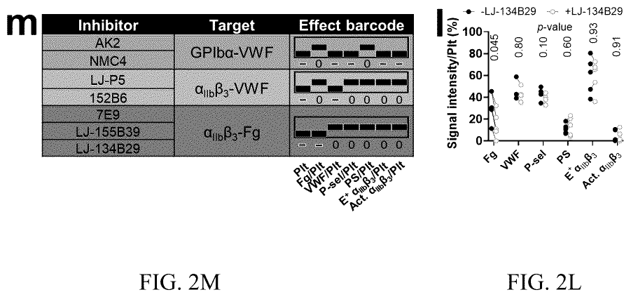

Delineating the respective contribution of different receptor-ligand interactions. Biomechanical platelet aggregation is mainly mediated by a mechanosensing axis on the platelet surface composed of two mechanoreceptors: GPIba and integrin αIIbβ₃. GPIba first binds to VWF to initiate platelet crosslinking, during which GPIba mechanosignaling induces integrin αIIbβ₃ intermediate activation (E⁺Act.). The activated integrin αIIbβ₃ binds to its ligands VWF and Fg to reinforce the platelet crosslinking process and also trigger its own further activation towards the fully activated state (E⁺Act.⁺) (Chen et al., Nature materials 18, 760-69, 2019). To investigate how the above platelet-crosslinking mechanisms, namely, GPIba-VWF, integrin αIIbβ₃-VWF, and integrin αIIbβ₃-Fg interactions, respectively mediate the growth, composition and activation status of biomechanical thrombi, blood was treated with a panel of highly specific inhibitory mAbs to inhibit them one at a time (FIG. 2A). AK2 and NMC4 both inhibit GPIba-VWF interaction, with AK2 targeting GPIba, and NMC4, previously shown to have anti-thrombotic effects (Kanaji et al., Blood advances 2, 2522-32, 2018), targeting VWF Al domain (VWFA1) which binds to GPIba (Kanaji et al., Blood advances 2, 2522-32, 2018; Yuan et al., The Journal of biological chemistry 274, 36241-51, 1999). LJ-P5 and 152B6 both inhibit integrin αIIbβ₃-VWF interaction, with LJ-P5 blocking integrin αIIbβ₃ binding to VWF but not Fg (De Marco et al., The Journal of clinical investigation 77, 1272-77, 1986), and 152B6 blocking VWF binding to integrin αIIbβ₃ but not GPIba (Berliner et al., The Journal of biological chemistry 263, 7500-05, 1988). 7E9, LJ-155B39 and LJ-134B29 inhibit integrin αIIbβ₃-Fg interaction by respectively blocking one of the three integrin-binding sites in Fg: γ408-411 (AGDV), Aa95-98 (RGDF) and Aa572-575 (RGDS) (Lengweiler et al., Biochemical and biophysical research communications 262, 167-73, 1999; Felding-Habermann et al., The Journal of biological chemistry 267, 5070-77, 1992).

Single fluorescence imaging was first used to measure the dose-dependency of the above mAbs in inhibiting thrombogenesis (FIG. 2B-2E). Only AK2 and NMC4, but not the other mAbs, eliminated thrombogenesis (FIG. 2C-2E), which agrees with previous findings that GPIba-VWF interaction serves as the initiator of biomechanical platelet aggregation, without which integrin αIIbβ₃ cannot be activated for platelet crosslinking (Nesbitt et al., Nature medicine 15, 665-73, 2009; Chen et al., Nature materials 18, 760-69, 2019). At high concentrations, both LJ-P5 and 152B6 reduced the thrombus size to <20% (FIG. 2D), and the cocktail of 7E9, LJ-155B39 and LJ-134B29 also reduced the thrombus size to ˜5% (FIG. 2E), indicating comparably important roles of integrin αIIbβ₃-VWF and αIIbβ₃-Fg interactions. When added alone, 7E9 achieved a strong inhibitory effect comparable to the cocktail, which corroborates the well-acknowledged primary role of AGDV in Fg for integrin αIIbβ₃ binding (Rooney et al., Blood 92, 2374-81, 1998; Liu et al., Biochimica et biophysica acta 1343, 316-26, 1997). However, LJ-155B39 and LJ-134B29 also manifested considerable inhibition (FIG. 2E).

Half-maximal inhibitory concentrations (IC50) were acquired for these mAbs via model fitting, which were then used in thrombus profiling. Both AK2 and NMC4 significantly decreased VWF, P-selectin, and E⁺and Act. αIIbβ₃ levels in the thrombus (FIG. 2F, 2G). In comparison, LJ-P5 and 152B6 only reduced VWF enrichment, while 7E9, LJ-155B39, and LJ-134B29 only reduced Fg enrichment; neither set of mAbs inhibited PS exposure, P-selectin expression, or integrin αIIbβ₃ activation (FIG. 2H-2L). None of the above mAbs affected the average signal intensity of SZ22-FITC, ruling out the possibility that the reduced VWF and Fg signals were due to increased platelet density. Altogether, our results indicate that different platelet-crosslinking mechanisms cooperatively mediate biomechanical thrombogenesis, with each having a distinct focus in their contribution to the thrombus composition and activation status.

To succinctly express the effects of different factors on biomechanical platelet aggregation, we created an “effect barcode” system with 7 columns each corresponding to one dimension of the thrombus profile. A positive, neutral, or negative effect of a factor on a dimension is respectively represented by a bar at the top, middle or bottom of the column, also numerically expressed as ‘+’, ‘0’, or ‘−’. Using this system, the effects of AK2 and NMC4 on the thrombus profile are both summarized as [−0−0−], those of LJ-P5 and 152B6 as [−0−00 0 0], and those of 7E9, LJ-155B39 and LJ-134B29 as [−−0 0 0 0 0] (FIG. 2M).

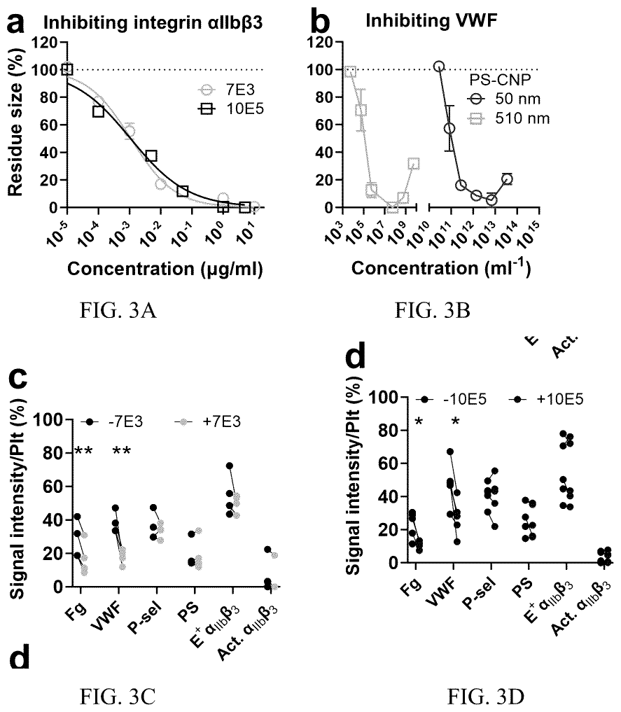

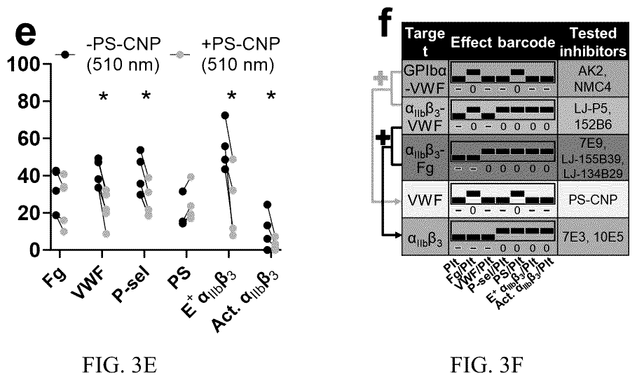

Identifying an ‘addition rule’ in the effect barcode system. Intrigued by how different receptor-ligand interactions synergize in mediating biomechanical thrombogenesis, we tested inhibitors with combinational effects. 7E3 (prototype of the antiplatelet abciximab) and 10E5 are mAbs that block integrin αIIbβ₃ binding to both Fg and VWF(Chen et al., Nature materials 18, 760-69, 2019; Coller, The Journal of clinical investigation 76, 101-08, 1985; Kaul et al., Blood 95, 368-374, 2000). Unlike specific inhibitors of integrin αIIbβ₃-VWF or αIIbβ₃-Fg, both 7E3 and 10E5 eliminated thrombogenesis at high concentrations (FIG. 3A). At IC50, both mAbs reduced Fg and VWF levels in the thrombus without affecting platelet activation markers, rendering an effect barcode of [−−−0 0 0 0] (FIG. 3C,3D). Interestingly, this barcode equals the add-up of those of integrin αIIbβ₃-VWF([−0−0 0 0 0]) and αIIbβ₃-Fg ([−−0 0 0 0 0]) inhibitors (FIG. 3F).

Negatively charged nanoparticles inhibit platelet aggregation at high shear rates due to their inhibition of VWF extension and, therefore, VWF-platelet interactions (Griffin et al., Biomicrofluidics 12, 042210, 2018). We tested two sizes of polystyrene negatively charged nanoparticles (PS-CNP) (50 and 510 nm), both showing biphasic dose-dependency in thrombus inhibition (FIG. 3B), consistent with the original report (Griffin et al., Biomicrofluidics 12, 042210, 2018). A concentration that decreases the thrombus size by ˜50% was estimated for the 510 nm PS-CNP to perform thrombus profiling (FIG. 3E), which derived an effect barcode of [−0−0−]. Again, this barcode equals the add-up of those of GPIba-VWF([−0−0−−]) and integrin αIIbβ₃-VWF([−0−0 0 0 0]) inhibitors (FIG. 3F). The above results demonstrate that the mathematical addition rule applies to the effect barcode system. This addition rule will be further validated below in drug-disease interactions.

Multi-dimensional thrombus profile abnormality in hypertension and aging. Aging and hypertension are two well-known strong risk factors for thrombosis (Wilkerson and Sane, Seminars in thrombosis and hemostasis 28, 555-68, 2002; Tsao et al., Circulation 145, e153-e639, 2022). To test the performance of our assay in identifying risks of arterial thrombosis, we first compared the thrombus size of healthy adults at different ages and identified that older ages (≥50) significantly increase the thrombus size (FIG. 4A). Furthermore, we tested blood samples from primary hypertension patients, which formed much larger biomechanical thrombi than healthy young subjects (FIG. 4B). By fitting the “thrombus size versus time” curves with the sigmoidal model, it was observed that unlike the growth of healthy young subjects' thrombi which approached a plateau at ˜400 s, hypertension patients' thrombi remained in the rapid development phase until ˜500 s, also indicating a clear prothrombotic tendency (FIG. 4B). Characterizing the thrombus profile revealed that aging and hypertension, either alone or together, significantly increased the thrombus size, Fg level as well as integrin αIIbβ₃ activation in the thrombi, rendering the same effect barcode of [++000++] (FIG. 4C). Two-way ANOVA with variance heterogeneity identified a bi-directional cooperation between hypertension and aging in increasing the thrombus size and E⁺αIIbβ₃ level, indicating strong synergy between these two risk factors (FIG. 4D). None of the above abnormalities was contributed by platelet density changes in the thrombus or hematocrit changes or platelet count increase in the blood.

We then evaluated the inter-correlation of the different biomarkers and their performance in distinguishing different cohorts. To address the scattering patterns of the signal intensities (FIG. 4C), which is likely due to inter-individual variability, multiple statistical analyses were performed for cross-check. Firstly, by using linear regression model, Spearman rank correlation coefficient (Charles, The American Journal of Psychology 15, 72-101, 1904) and Kendall's tau correlation coefficient (Kendall, Biometrika 30, 81-93, 1938), a positive correlation was consistently identified between thrombus size and Fg, E⁺αIIbβ₃ and Act. αIIbβ₃ levels but not the other factors (FIG. 4E), with E⁺αIIbβ₃ being the strongest correlating factor. Secondly, among all markers, E⁺αIIbβ₃ has the best performance in separating healthy young from hypertensive and/or older age groups (FIG. 4E), with specificity and sensitivity respectively reaching 86% and 85%, comparable to the performance of thrombus size. The consistency of E⁺αIIbβ₃ level with thrombus size in group separation also reached 81%. Altogether, these results unraveled intensified biomechanical thrombogenesis and multi-dimensional thrombus profile abnormality associated with hypertension and aging and suggest E⁺αIIbβ₃ as a potential biomarker for intensified biomechanical thrombogenesis.

Most hypertension patients enrolled in this study had their blood pressure well controlled by medication (systolic/diastolic <140/90 mmHg, respectively) and had hemoglobin A1C(HbAlC), body mass index (BMI) and cholesterol levels within the healthy range (FIG. 4F). Furthermore, neither the size nor the E⁺αIIbβ₃ level of these patients' thrombi has a significant correlation with the disease duration, systolic or diastolic blood pressure, or the sum of the two, or the patients' HbAIC level, BMI, total cholesterol, low-density lipoprotein cholesterol (LDL-C), high-density lipoprotein cholesterol (HDL-C) or triglyceride levels (FIG. 4F). Also, the thrombi of hypertensive subjects who have systolic and diastolic blood pressures and HbAlC, BMI, and cholesterol levels all in the normal ranges still have larger sizes and higher E⁺αIIbβ₃ levels than healthy young subjects, regardless of aging. These results indicate that hypertension can independently cause intensified biomechanical thrombogenesis and thrombus profile abnormality even with relatively short disease duration and effective antihypertensive medication. However, we cannot exclude the likelihood that poorly controlled blood pressure, diabetes (high HbAlC level), obesity (high BMI) or dyslipidemia (abnormal cholesterol levels) can have extra contributions to the thrombus profile abnormality, especially considering that the latter three diseases are known risk factors of CVD.

Next, we inspected whether demographics other than age affect the thrombus profile. Within healthy young as well as hypertensive and/or older subjects, no significant difference in the thrombus size or E⁺αIIbβ₃ level was found between males and females or among different races/ethnicities (FIG. 4G-4L). Seemingly in discrepancy with previous reports of a higher prevalence of CVD in males than in females and slight prevalence differences in different ancestries, these results corroborates more careful cohort studies demonstrating that the correlation of gender and ancestry with thrombotic risks is mainly due to the differential prevalence of social determinants of health and cardiovascular risk factors (Tsao et al., Circulation 145, e153-e639, 2022; Safford et al. Jama 308, 1768-74, 2012; Colantonio et al., Circulation 136, 152-66, 2017).

Due to size variations, different human arteries and arterioles have distinct Reynolds numbers (affecting flow patterns such as laminar versus turbulent) and shear rates in the blood flow (Mahalingam et al., Cardiovasc Diagn Ther 6, 208-20, 2016; Ku, Annu. Rev. Fluid Mech. 29, 399-434, 1997), together resulting in a certain extent of diversification in the shear stress. However, changing the perfusion rate in our assay from 18 to 13.5, 27 and 36 μl/min (respectively changing the shear stresses to 0.75, 1.5 and 2 times of the original) did not significantly affect the thrombus profiling outcome, wherein significantly larger thrombus size and higher E⁺and Act. αIIbβ₃ levels, marginally higher Fg level but comparable VWF, P-selectin and PS levels were consistently observed in the thrombi of hypertensive young subjects than healthy young subjects (FIG. 7). These results validated that our assay assesses the general shear-driven platelet “aggregatability” of blood samples.

Hypertension causes hyperactivity of GPIba-integrin αIIbβ₃mechanosensing axis. We previously identified that the intermediate activation state of αIIbβ₃ integrin with an extended-close conformation (E⁺Act.⁺) plays a crucial role in biomechanical platelet aggregation (Chen et al., Nature materials 18, 760-69, 2019). Thus, the over-expressed E⁺αIIbβ₃, predominantly E⁺Act.⁺ αIIbβ₃ (FIG. 4C) in the thrombi of hypertensive patients should directly contribute to the intensified biomechanical thrombogenesis. We hypothesize that the E⁺αIIbβ₃ over-expression is likely due to (1) hyperactivity in GPIba, with triggers stronger mechanosignaling for integrin activation (Chen et al., Nature materials 18, 760-69, 2019), and/or (2) integrin αIIbβ₃ pre-activation in the patients' body. To test these two hypotheses, we used four complementary approaches to investigate the activities of GPIba and integrin αIIbβ₃ in hypertension patients.

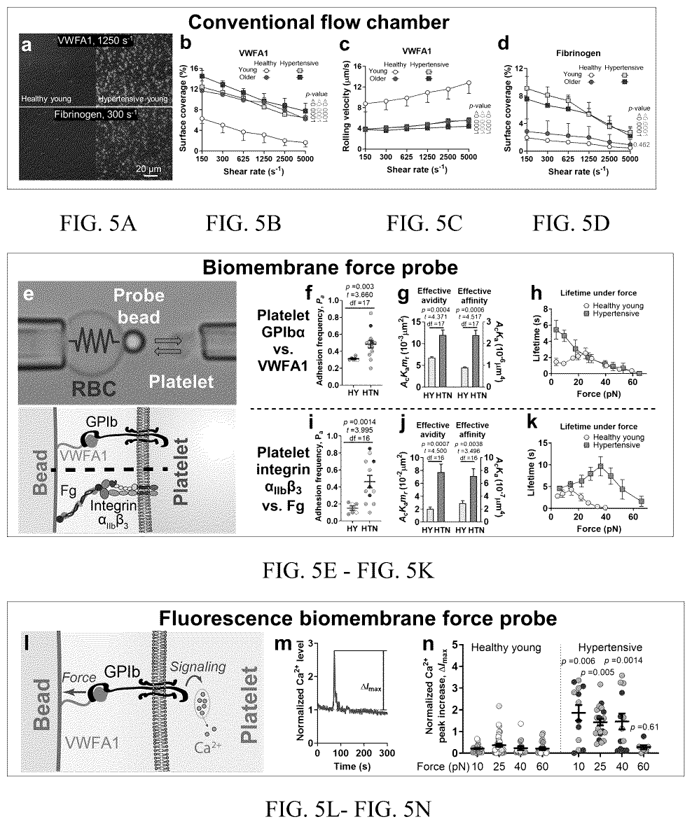

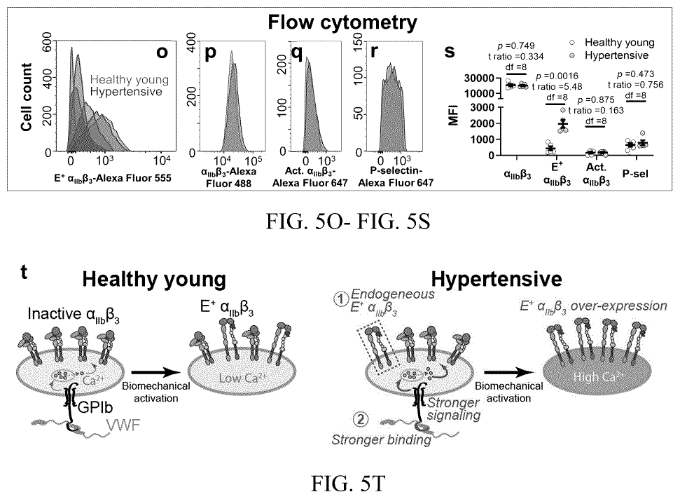

Firstly, conventional laminar flow chamber assay was used to measure the overall ligand binding activity of the two receptors. Unlike the stenosis assay, here the channels adopt a plain surface pre-coated with VWFA1 or Fg to engage GPIba and integrin αIIbβ₃, respectively. Plasma in the blood was depleted and replaced with buffer to prevent the interference of endogenous VWF and Fg and avoid platelet aggregation. By perfusing blood through the channels under varied shear rates, it was shown that platelets from hypertensive young, hypertensive older and healthy older subjects all achieved much higher surface coverage and slower rolling on VWFA1 than healthy young group (FIG. 5A-5C). On the other hand, only hypertensive young and hypertensive older groups achieved high surface coverage on Fg, while healthy young and healthy older groups were low (FIG. 5A-5D). These results indicate that both hypertension and aging cause GPIba hyperactivity, but only hypertension induces hyperactivity in integrin αIIbβ₃ at the same time. Considering that the activities of GPIba and integrin αIIbβ₃in hypertensive young and hypertensive older subjects were comparable (FIG. 5B-5D), all mechanistic studies below combined young and older hypertensive subjects into a single cohort to compare with healthy young subjects. However, this does not exclude the possibility that aging can influence hypertensive patients' GPIba and integrin αIIbβ₃ as a secondary factor, which shall be inspected in later studies.

Secondly, a single-molecule force spectroscopy technique, biomembrane force probe (BFP) (Chen et al., Journal of visualized experiments, e52975, 2015), was used to measure the ligand binding of single platelets. A micropipette-aspirated biotinylated human red blood cell (RBC) was used as an ultrasensitive force transducer, and a probe bead co-functionalized with streptavidin and VWFA1 or Fg was glued to the RBC apex. A platelet was aspirated by an opposing micropipette and driven to repeatedly contact the bead, which induced adhesion events to measure the receptor-ligand binding kinetics (FIG. 5E). Adhesion frequency assay was first deployed to enumerate the absence or presence of adhesion events after long contacts to calculate the steady-state adhesion frequency, Pₐ (Chesla et al., Biophysical journal 75, 1553-72, 1998). The Pa of hypertensive subjects' platelets adhering to the same batch of VWFA1 and Fg beads were significantly higher than healthy young (FIG. 5F,51), reflecting a significantly higher effective avidity (ligand-binding capability of each unit of platelet surface area) of both GPIba and integrin αIIbβ₃ (FIG. 5G,5J), which is consistent with the platelets' enhanced capability of engaging VWFA1 and Fg in the flow chamber (FIG. 5A-5D). Dividing effective avidities by the receptors' surface density showed that the average effective affinity of GPIba and integrin αIIbβ₃ on the hypertensive subjects' platelets were also significantly enhanced (FIG. 5G,5J right). Then, the BFP force-clamp assay was used to measure the stability of single GPIba-VWFA1 and integrin αIIbβ₃-Fg bonds under force. This was achieved by adjusting the contact time between the bead and the platelet to achieve Pa ˜20%, thereby realizing a ˜90% probability of single bonds (Chesla et al., Biophysical journal 75, 1553-72, 1998). The GPIba-VWFA1 bond lifetime of hypertensive subjects' platelets manifested a ‘slip bond’ instead of a triphasic ‘slip-catch-slip’ trend seen on healthy young subjects' platelets (Ju et al., Thrombosis research 136, 606-12, 2015), resulting in a substantial prolongation of bond lifetimes under forces <20 pN (FIG. 5H). On the other hand, hypertension caused a substantial rightward and upward shift of the integrin αIIbβ₃-Fg catch bond (Chen et al., Nature materials 18, 760-69, 2019), so that the peak force increased from ˜15 to ˜35 pN, the peak lifetime increased from ˜5 to ˜10 s, and the force range where lifetime events were observable was widened from 0-40 to 0-65 pN (FIG. 5K). Notably, this lifetime curve from hypertensive subjects' platelets also resembles healthy young subjects' E⁺Act.⁺ integrin αIIbβ₃-Fg lifetime curve characterized previously (Chen et al., Nature materials 18, 760-69, 2019). Altogether, our BFP results indicate that hypertension increases not only the avidity, but also the affinity and force-regulated ligand binding strength of GPIba and integrin αIIbβ₃.

Thirdly, we combined fluorescence imaging with BFP (fBFP) to study whether the increased affinity and ligand binding strength of GPIba in hypertension patients can result in stronger mechanosignaling to better induce integrin αIIbβ₃ activation. Platelets pre-loaded with Ca²⁺ dye (Fura-2) were repeated stimulated by a VWFA1-coated bead in force-clamp cycles at a fixed 2-s contact time for 5 min (FIG. 5L), while the normalized intraplatelet Ca²⁺ level was monitored (FIG. 5M). Agreeing with our hypothesis, hypertension patients' platelets fluxed stronger Ca²⁺ signals-reflected by higher Ca²⁺ peak increase—than healthy young subjects' platelets under a wide force range (FIG. 5N). Unlike healthy young subjects' platelets where the Ca²⁺ signal first increases and then decreases with clamping force, mirroring their lifetime's ‘catch-slip’ trend, the Ca²⁺ signal of hypertension patients' platelets manifested a gradual decline with clamping force, also consistent with the shape of their GPIba-VWFA1 lifetime slip bond (FIG. 5N). This corroborates our previous finding that the intensity of GPIba mechanosignaling, manifested by both Ca²⁺ signaling and integrin αIIbβ₃ activation, heavily relies on the duration of force pulling on GPIba (Ju et al., eLife 5, e15447, 2016).

Fourthly, flow cytometry was used to investigate whether the αIIbβ₃ integrins on hypertension patients' platelets are pre-activated. While similarly high expression of integrin αIIbβ₃ and baseline expression of Act. αIIbβ₃ and P-selectin were detected on the platelets of healthy young and hypertensive subjects, the expression of E⁺αIIbβ₃ in the hypertensive group was found to be much higher than in the healthy young group (FIG. 50-5S). Although hypertension patients' platelets are slightly larger than healthy young subjects′, a positive correlation between E⁺αIIbβ₃ signal and platelet volume was found only in the hypertensive group but not healthy young group. These results indicate that hypertension patients' platelets are pre-activated, with integrin αIIbβ₃ up-regulated to the intermediate activation state (E⁺Act.) and minimal P-selectin expression.

Altogether, our results indicate that two mechanisms work in parallel to induce E⁺αIIbβ₃ over-expression in the biomechanical thrombi of hypertensive patients (FIG. 5T): (1) some αIIbβ₃ integrins already adopt a native E⁺status rather than remaining inactive as on healthy platelets; and (2) hyperactive GPIba triggers stronger mechanosignaling upon VWF binding, inducing more αIIbβ₃ integrins to undergo E⁺activation than on healthy platelets.

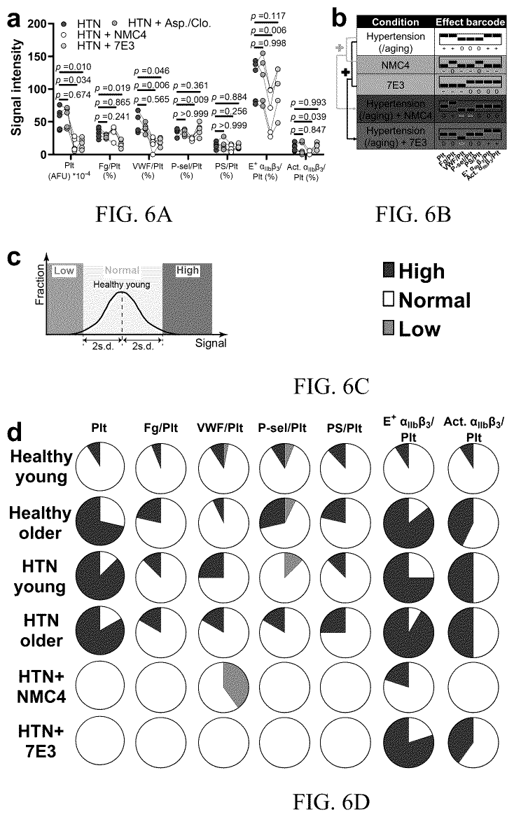

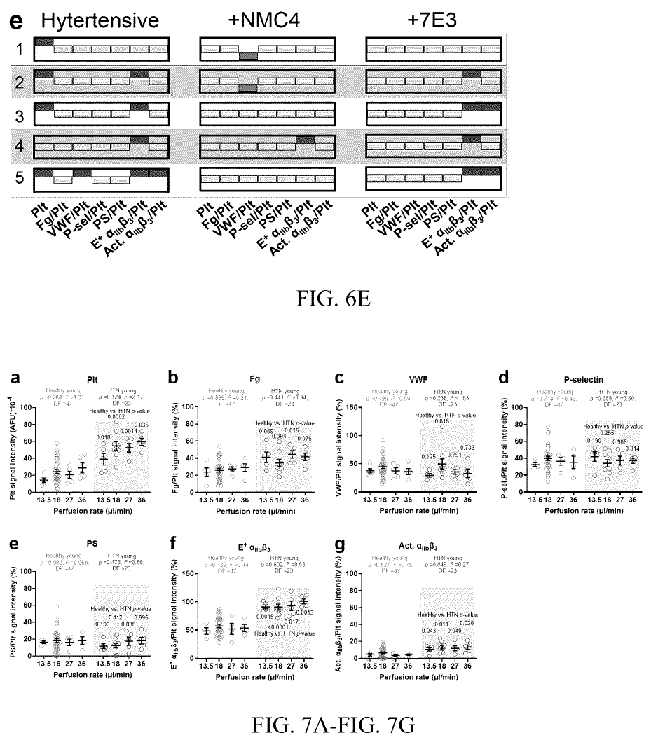

Expanding the addition rule to drug-disease interactions. Using the thrombus profiling assay, we tested how anti-thrombotic inhibitors affect the thrombus profile of hypertension patients. Consistent with our results on healthy subjects, the combination of aspirin and clopidogrel at twice of human plasmatic concentrations (Arrebola et al., J Cardiovasc Pharmacol 43, 74-82, 2004) showed no effect on hypertension patients' thrombi (FIG. 6A). In contrasts, at IC50, NMC4 reduced the thrombus size and E⁺and Act. αIIbβ₃ expression to healthy levels, but also lowered VWF and P-selectin levels unaffected by hypertension (FIG. 6A). The resulting effect barcode, [0+−−0 0 0], equals the add-up of those of NMC4 and hypertension (FIG. 6B). Similarly, adding 7E3 to hypertension patients' blood resulted in an effect barcode of [00−00++], equaling the add-up of the effect barcodes of 7E3 and hypertension (FIG. 6A,6B). These results indicate that the addition rule of the barcode system can also be applied to predict drug-disease interactions. Neither NMC4 nor 7E3 completely corrected the effect barcode of hypertension, with 7E3 even incapable of suppressing the integrin αIIbβ₃ over-activation, implying a treatment mismatch between the inhibitors and the patients.

Inter-individual variability in personal thrombus barcodes. Lastly, to evaluate the normality and abnormalities of individuals' thrombus profiles, we created the concept of “personal thrombus barcodes”. From the thrombus profiles of healthy young subjects, values of each dimension were fitted to a Gaussian distribution, of which the mean±2s.d. (˜95% confidence interval) was defined as the reference range (‘0’), and values lower or higher were defined as abnormally low (‘−’) and high (′+′), respectively (FIG. 6C).

Applying this system to healthy young subjects rendered all dimensions of thrombus profiles being dominated by normal values, with only very small fractions being abnormally low or high, which is consistent with the definition of the reference ranges (FIG. 6D). In contrast, much larger proportions of healthy older, hypertensive young, and hypertensive older subjects had large thrombi and high E⁺and Act. αIIbβ₃ levels, with the fraction having high Fg levels also moderately higher (FIG. 6D). Most of these subjects (26/36) have high values in thrombus size and E⁺αIIbβ₃ level, yet 3 subjects with abnormally large thrombi have a normal E⁺αIIbβ₃ level. A high Act. αIIbβ₃ level was observed in half of the subjects with large thrombi (14/29), but also in 2 subjects with normal thrombi. Most subjects in these three groups (23/36) have abnormal VWF, P-selectin and PS levels, which may or may not co-exist with high values of thrombus size and E⁺αIIbβ₃ level. Of all the 69 subjects, a total of 30 different personal thrombus barcodes were identified. Overall, the above results indicate strong inter-individual variability in the personal thrombus barcode that cannot be ascribed to disease and aging, and demonstrate obvious decoupling of the different dimensions in the thrombus profile. Notably, we repeated our test on 14 randomly picked subjects after different time intervals (from 2 weeks to 9 months). Among a total of 21 re-tests, only 2 showed changes in the personal thrombus barcodes, which were associated with the longest time intervals (7 and 9 months, respectively). This reflects high reliability of our assay and indicates that the personal thrombus profiles of individuals are relatively stable but can still vary over time.

Inter-individual variability was also observed in the subjects' responses to anti-thrombotic inhibitors. While NMC4 effectively corrected the size and E⁺αIIbβ₃ level in most hypertension patients' thrombi, it did not uniformly modify all their personal thrombus barcodes, but instead produced three different barcodes: [0 0 0 0 0 0 0], [0 0−0 0 0 0], and [0 0 0 0 0+0] (FIG. 6D,6E). Similar diversification was also found in 7E3, despite its consistent negative effect on the thrombus size and neutral effect on E⁺αIIbβ₃ level (FIG. 6D,6E). These diversifications cannot be completely ascribed to differences in the patients' original personal thrombus barcodes (FIG. 6E).

Hemodynamics in humans: It was shown by simulation that coronary arteries can reach a high Reynolds number and could develop turbulence with 70% or above stenosis (Mahalingam et al., Cardiovasc Diagn Ther 6, 208-20, 2016), and in vivo experiments demonstrated that turbulence can have a positive effect on thrombus formation (Stein and Sabbah, Circulation research 35, 608-14, 1974). It was postulated that turbulence contributes to arterial thrombosis by providing extra mechanical forces (Ouriel et al., Journal of vascular surgery 14, 757-62, 1991). On the other hand, how flow contributes to thrombosis is unclear. We calculated the Reynolds number in our channel at the site of stenosis to be 3.73, which is certainly lower than that in large human arteries, e.g., aorta. However, it is important to note that the size of blood vessels also varies tremendously between different animal species: the diameter of carotid artery is ˜0.7 mm in mice and ˜1 mm in rats but can reach ˜8 mm in human (Panteleev et al., Journal of thrombosis and haemostasis: JTH 19, 588-95, 2021). Thus, the Reynolds numbers in the arterial blood flow of mice and rats versus human are also greatly different. Yet, this does not impede the wide application of mouse and rat models for providing valuable information regarding vascular health and diseases with close human relevance and for evaluating anti-thrombotic strategies and drugs.

Furthermore, due to the great size diversification of different human arteries (diameter: roughly 1-20 mm) and arterioles (diameter: 15-240 μm), the Reynolds number of the blood flow in different arteries and arterioles in fact varies drastically, ranging from ˜1 to approximately 4,000. Even within different carotid arteries, the Reynolds number can range from tens to 4,000 (Mahalingam et al., Cardiovasc Diagn Ther 6, 208-20, 2016; Ku, Annu. Rev. Fluid Mech. 29, 399-434, 1997). Considering that a Reynolds number of 2,000 is generally considered the threshold for determining laminar versus turbulent flow, and a Reynolds number in the scale of tens would start to allow flow separation, the high variability of Reynolds number in human arteries and arterioles indicates high variability in the flow pattern of blood in different arteries and arterioles. Yet, thrombosis can technically occur in all arteries and arterioles. Therefore, it is impossible to use a single flow pattern to represent all the hemodynamic conditions in arterial thrombosis or use a single setup to recapitulate hemodynamic conditions in all arteries and arterioles.

While it is true that the Reynolds number in microfluidic systems is lower compared to large human arteries due to differences in lumen size our device is mainly designed to focus on the biorheological conditions most critical for platelet aggregation and thrombus formation, namely, high shear flow. The device recapitulates the hemodynamic environment of typical arteries and arterioles in the sense that, the wall shear stress (WSS) it creates at the stenosis site is comparable to that in human arterioles and mouse arteries, as well as in large human arteries during systole (Panteleev et al., Journal of thrombosis and haemostasis: JTH 19, 588-95, 2021; Yakusheva et al. Blood advances 6, 4834-46, 2022). Although this wall shear rate is higher than the average wall shear rate of large human arteries (250-1,000 s-1), its resulted extra high shear rate at the site of stenosis would create extra shear stress that to some extent compensates the missing shear stress that ought to be provided by turbulence in large arteries. The Reynolds numbers in channels can be compared with the same cross-sectional area but different shapes, which are within the same scale. At the inlet site with no stenosis, the Reynolds number is 0.73 for a rectangular channel (adopted by our device) and 0.71 for a circular (shape of arteries and arterioles) channel. At the site of stenosis, the Reynolds number is 2.01 for a rectangular channel and 1.15 for a circular channel. These comparisons confirm that the shape of the channel does not incur a great change in the Reynolds number.

Creating turbulent flow in microfluidic channels either requires extremely high perfusion rates which are non-physiological or requires the channel geometry to contain highly non-smooth components (Wang et al., Lab on a chip 14, 1452-58, 2014). Considering that turbulence contributes to arterial thrombosis by providing extra mechanical forces (Ouriel et al., Journal of vascular surgery 14, 757-62, 1991), we reason that if we can confirm that an increase or decrease of shear stress within a certain range does not compromise the capability of our setup in assessing the shear-driven platelet “aggregatability” of blood samples, then our assay has general biorheological relevance. Therefore, we performed new experiments with varied perfusion rates to change the shear stress inside the channel. Our results showed that lowering the blood perfusion rate from 18 to 13.5 μl/min or increasing it to 27 and 36 μl/min (which respectively changed the shear stresses to 0.75, 1.5 and 2 times of the original) did not significantly affect the thrombus profiling outcome (FIG. 7). Furthermore, despite variations in the perfusion rate, we could consistently observe significantly larger thrombus size (FIG. 7A) and higher E⁺and Act. αIIbβ₃ levels (FIG. 7F,7G), marginally higher Fg level (FIG. 7B) but comparable VWF, P-selectin and PS levels (FIG. 7C-7E) in the thrombi of hypertensive young subjects than healthy young subjects. These results validated our hypothesis, and confirmed the robustness of our assay.

Barcode: That “platelet aggregation is all that is needed to block a vessel”, or in other words, “size is the only important parameter in thrombi”, represents the mainstream viewpoint in the current thrombosis field. However, we argue that this viewpoint may not be necessarily correct. Around the year 2000, a series of oral antagonists of integrin αIIbβ₃ were developed. which were expected to represent “the dawn of a new era in anti-thrombotic therapy, the era of αIIbβ₃ antagonism” (Topol et al., Lancet 353, 227-31, 1999). However, as documented by multiple phase III trials, these integrin αIIbβ₃ antagonists (e.g., orbofiban) significantly increased patient mortality than placebo by enhancing the risk of myocardial infarction, despite their high potency in inhibiting platelet aggregation (Topol et al., Lancet 353, 227-31, 1999; Chew et al., Circulation 103, 201-06, 2001). This led to the discontinuation of these integrin αIIbβ₃ antagonists for anti-thrombotic usage. It was till years later that researchers started to find that the ‘toxicity’ associated with these agents (Chew et al., Circulation 103, 201-06, 2001) was due to their effect of stabilizing integrin αIIbβ₃ in the active open conformation (Bougie et al., Blood 119, 6317-25, 2012), which corresponds to the E⁺Act.⁺ status defined in our manuscript. The activation of integrin αIIbβ₃ would lead to platelet activation (Cox et al., Journal of the American College of Cardiology 36, 1514-19, 2000), which is featured by granule release and the expression of more, active integrin αIIbβ₃ that may cause relapse of thrombosis once the dose of the αIIbβ₃ antagonist declines in the plasma. In other words, although these αIIbβ₃ antagonists temporarily silence the platelets in patients, they also convert these platelets into prothrombotic “time bombs”. The αIIbβ₃ antagonists also causes thrombocytopenia as another side-effect due to the production of autoantibodies against the epitopes only exposed in E⁺Act.⁺ integrins (Bougie et al., Blood 119, 6317-25, 2012; Bosco et al., Journal of thrombosis and haemostasis: JTH 3, 1109-10, 2005). Learning from the failure of these αIIbβ₃ antagonists, a chemical principle was recently developed to develop better anti-thrombotic candidates that lock integrin αIIbβ₃ in the inactive state. Altogether, the above works and findings strongly indicate that the size is not the only important parameter of a platelet aggregate or a thrombus. Instead, the multi-dimensional characteristics of thrombi, summarized as a ‘profile’ or a ‘barcode’ in our system, should be viewed carefully and comprehensively.

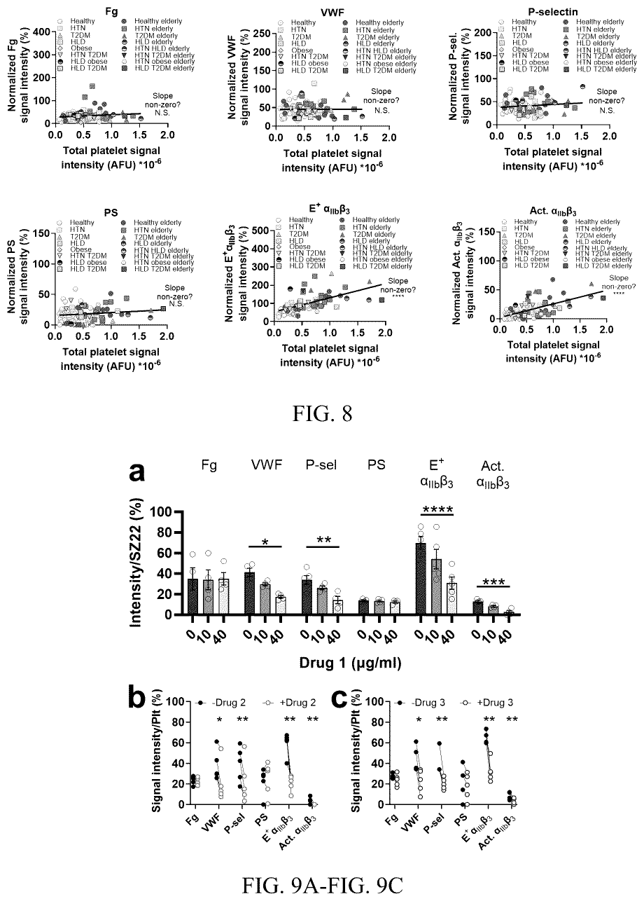

Following the above idea, the barcode system is useful in three aspects: (1) Anti-thrombotic drug evaluation. The above integrin αIIbβ₃ antagonists are hardly accessible, considering that they were discontinued more than 20 years ago. However, we postulate that they correspond to an effect barcode of [−−−00++], based on the barcode of 7E3 and 10E5 ([−−−0 0 0)]) and the fact that they can induce integrin αIIbβ₃ activation. Thus, if we find drug candidates in future that also show a ‘+’ sign in the last two dimensions of their effect barcodes, we would be alarmed that they will likely cause similar life-threatening effects. Although no previous research has reported any anti-thrombotic agents that would increase Fg, VWF, P-selectin or PS level in the thrombus, it is reasonable to suspect that such effects would also bring extra risks to patients' health. (2) Patient prothrombotic status characterization. People carrying risk factors of arterial thrombosis can have abnormalities in their thrombus barcode. Detecting these abnormalities would provide valuable information regarding the patient's prothrombotic status than only checking the size of the thrombus. Taking the study of hypertension in our work as an example, it was the identification of hypertension patients' thrombus barcode, [++0 0 0++], with a high E⁺αIIbβ₃ expression, that inspired us to eventually uncover the hyperactivity of GPIba-integrin αIIbβ₃ mechanosensing axis as a new mechanism underlying the high risk of CVD in hypertension patients. Only measuring the thrombus size would not provide us with any specific mechanistic cues. Similarly, if another thrombosis-associated disease or unhealthy habit (e.g., smoking) triggers an abnormally high level of Fg, VWF, P-selectin or PS level in the thrombus, then we would be advised that some mechanism(s) around this biomarker have malfunctions, contributing to the higher thrombotic risks. In addition to the hypertension and aging tested in the current work, we have tested patients with type II diabetes (T2DM), obesity or hyperlipidemia (HLD) and patients with two or more of these conditions, which consistently showed greater thrombus size and higher level of integrin αIIbβ₃ activation (FIG. 8) in these populations. (3) Personalized drug selection. Acquiring the thrombus barcode of a patient may also help clinicians deicide what drug or combination of drugs can reach higher efficacy and less side-effect in this patient. For example, NMC4 and 7E3 both inhibited the intensified biomechanical thrombogenesis of hypertension patients effectively. However, only NMC4 but not 7E3 can correct the over-activation of integrin αIIbβ₃, indicating that abciximab (a derivative of 7E3 approved for clinical use) may not be the optimal drug for reducing the thrombotic risks in hypertension patients.

Besides the above advantages, the barcode system is useful in one more aspect which is related to drug screening and mechanism discovery. As we stated in the original manuscript, an interesting attribute regarding the barcode system is that, “the effect barcode of each anti-thrombotic agent is dictated by its target rather than its pharmacological design”. Effective barcodes can be used as identifiers of different contributing factors of biomechanical platelet aggregation, which allows the assay to pinpoint the functional context of new mediators and mechanisms, tackling the limitation of existing similar assays in providing mechanistic implications of arterial thrombosis. Alias: Drug 1, Drug 2, and Drug 3. Drug 1 is a small peptide known to inhibit VWFA1 binding to GPIba. Indeed, as we characterized, Drug 1 manifests an effect barcode of [−0−0−] (FIG. 9A), identical to AK2 (GPIba inhibitor) and NMC4 (VWFA1 inhibitor) studied in our current work. Drug 2 is a small-molecule compound. We used our thrombus profiling assay for drug screening and identified ˜15 hits from a compound library, with Drug 2 being one of the most potent. Drug 2 manifests an effect barcode of [−0−0−] (FIG. 9B), suggesting its target to be GPIba-VWFA1 interaction. Indeed, our nano differential scanning fluorimetry and ELISA results confirmed that Drug 2 can specifically bind to VWFA1 and inhibit GPIba-VWFA1 interaction (data not shown). Drug 3 is a molecule naturally existing in the blood. Our preliminary data on mouse experiments showed that it can effectively inhibit arterial thrombosis (data not shown). Considering that the effect barcode of Drug 3 is identical to AK2, NMC4, Drug 1 and Drug 2 (FIG. 9C), we are confident that its target should also be GPIba-VWFA1 interaction. Using the thrombus profiling assay and the barcode system can help us quickly identify the mechanism of action of unknown agents that manifest anti-thrombotic effects. In contrast, by only assessing the size of the platelet aggregate, we would merely know that this agent is anti-thrombotic but have no clue of why, and will have to look for its target from scratch.

A major challenge faced by the current anti-thrombotic treatments is antiplatelet resistance, meaning that the anti-thrombotic efficacy of the drug is compromised in certain patients (Comanici et al., Am J Cardiol 206, 191-99, 2023). Pathological conditions such as hypertension, diabetes, metabolic syndrome and aging not only exacerbate thrombotic risks but also foster antiplatelet resistance to conventional antiplatelets. Importantly, the antiplatelet drugs that are being widely used and constantly encountering resistance, such as aspirin and clopidogrel, all target “biochemical axes that control platelet aggregation”, such as thromboxane A2 (TXA2)-TXA2 receptor signaling axis, ADP-P2Y signaling axis, and thrombin-PAR signaling axis (where platelet activation are all triggered by soluble agonists). This indicates that effectively inhibiting platelet aggregation remains an unachieved goal, and solely targeting biochemical axes may not be able to resolve this problem. In such a context, our platform for the first time identified a hyperactive biomechanical axis that intensifies platelet aggregation but cannot be inhibited by conventional antiplatelets, which is both novel and clinically important, because it uncovers a new mechanism of antiplatelet resistance. We believe that effective inhibition of arterial thrombosis probably requires the inhibition of both biochemical and biomechanical mechanisms of platelet aggregation. Antiplatelet resistance is conventionally believed to be due to patients' lack of sensitivity to antiplatelets in inhibiting platelet amplification loops (Cattaneo, Thrombosis research 127 Suppl 3, S61-63, 2011). However, we found that biomechanical thrombogenesis is essentially “immune” to aspirin and clopidogrel (FIG. 6A). These, together with similar observations by others indicate a new mechanism of antiplatelet resistance: biomechanical platelet aggregation can mediate arterial thrombosis independent of platelet amplification mechanisms, and therefore the sole inhibition of platelet amplification loops allows thrombotic risks to persist by leaving biomechanical platelet aggregation active.

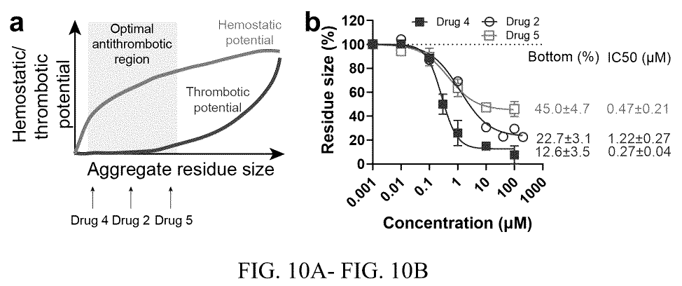

The platform does have the potential of evaluating bleeding risks. In a previous work, a microfluidic assay using a similar stenosis design was used to evaluate bleeding risks in patients with von Willebrand disease, and a negative correlation was identified between the aggregate size and bleeding score of these patients (Brazilek et al., Lab on a chip 17, 2595-2608, 2017). This work suggests the feasibility of using our thrombus profiling assay to test bleeding risks of subjects and to test the bleeding side-effect of anti-thrombotic drugs. We hypothesize that partial inhibitors that preserve a certain level of GPIba-VWFA1 binding capacity even under saturating concentrations may preserve normal hemostasis (FIG. 10A). Via screening, we have discovered 3 new VWFA1 inhibitors with such attributes (one being the Drug 2 mentioned above in FIG. 9B), which, at saturating concentrations, allow the generation of platelet aggregates with different residue sizes: from 10% to 25% to 50% (FIG. 10B).

### B. Methods

Study design. This study was designed to develop a translational multi-parametric platform that characterizes thrombogenesis in the arterial biomechanical settings. Based on previously developed stenosis flow chamber assay, we incorporated multi-fluorescence imaging to concurrently monitor the size, composition and activation status of the thrombus, together reporting a ‘thrombus profile’. After validating the feasibility of this thrombus profiling assay, we validated that the assay can distinguish the effects of anti-thrombotic antibodies with different targets. To assess whether the assay could detect higher risks of arterial thrombosis, we applied blood samples from hypertensive and older people and detected multi-dimensional thrombus abnormality that is featured by a significantly higher expression of activated integrin αIIbβ₃. Further analysis was conducted to explore the correlation between thrombus abnormality and the subjects' health conditions (blood pressure level, duration of hypertension, etc.) and demographics other than age. With prior evidence indicating a central role of activated integrin αIIbβ₃ in biomechanical thrombogenesis, we further employed 4 additional experimental approaches and investigated the molecular mechanisms underlying the integrin αIIbβ₃ over-activation in hypertension patients' thrombi. Lastly, we studied how different anti-thrombotic agents affect the thrombus profile of hypertension patients, and whether personal thrombus profiles have high inter-individual variability. Due to the nature of this study, randomization was not done. No statistical methods were used to predetermine sample sizes (n), which are indicated in the figures and legends. The investigators who collected blood samples and performed experiments and those who analyzed the experimental data were always separate. The investigators who analyzed the data were blinded to the detailed information of the subject until the analysis was complete.

Reagents. SZ22-FITC and P2-Alexa Fluor 488 (Beckman Coulter), Type I collagen, AK4-Alexa Fluor 647 and PAC-1-Alexa Fluor 647 (BioLegend), AK2, HIP-8-Alexa Fluor 488, Annexin V-Pacific Blue, Annexin V-Alexa Fluor 488, heparin, DiOC₆ (3) and Alexa Fluor 405, 555 and 647 conjugation kits (ThermoFisher Scientific), MBC 370.2 (Kerafast), fibrinogen (Innovative Research), NMC4, 2.2.9, LJ-P5, 152B6, LJ-155B39, LJ-134B29 and VWFA1 (MERU VasImmune), VWF monomer (Sino Biological), RU5 (Creative Biolabs) and PS-CNP beads (Bangs Laboratories) were purchased. 7E9, 7E3 and 10E5 were gifts from Barry S. Coller (Rockefeller University).

Human subjects. All procedures involving human subjects were approved by the Institutional Review Board of the University of Texas Medical Branch (protocol number: 22-0015) and the University of Sydney (ethics reference number: 2023/582). Informed consent was obtained from all subjects.

Number (n) and age (mean±s.d.) of subjects who participated in the thrombus profiling assay: healthy young: n=33, age=34.0±6.3; healthy older: n=14, age=62.1±8.9; hypertensive young: n=9, age=36.1±8.7; hypertensive older: n=13, age=60.2±9.4. All groups contained both male and female subjects with multiple races and both non-Hispanic/Latino and Hispanic/Latino ethnicities.

All hypertension patients were taking prescribed hypertension medications (e.g., prazosin, amlodipine, enalapril). Patients taking other medications or under the treatment for other diseases within 2 weeks before the blood draw were excluded from this study.

Blood collection, reconstitution and platelet isolation. For whole blood stenosis assay, blood was slowly drawn from the vein of a volunteer into a syringe pre-loaded with heparin (20 U/mL). In some control experiments, sodium citrate (4%) or EDTA (1.5 mg/ml) was used as the anticoagulant instead.

For laminar flow chamber assay, BFP assays and flow cytometry, blood was drawn into a syringe pre-loaded with ACD buffer. Then blood reconstitution was performed for laminar flow chamber assay to deplete plasma and reach a hematocrit of 45% and platelet count of 20,000 μL-1. Or, platelet isolation was performed for BFP assays and flow cytometry, with platelets finally resuspended in modified Tyrode's buffer (135 mM NaCl, 11.9 mM NaHCO₃, 2.9 mM KCl, 0.42 mM NaH₂PO₄, 10 mM Hepes, 5.5 mM dextrose, pH 7.4).

Microfluidic device preparation. Polydimethylsiloxane (PDMS) was applied on a silicon mold (1-μm resolution), which was heated at 75° C. for 1 h for curing, peeled off and cut into single pieces. Holes were drilled to create outlets and inlets. The devices then underwent plasma treatment and were bonded to glass coverslips.

Microfluidic stenosis assay. Microfluidic channels were coated with VWF monomer (2 μg mL-1) for 1 h. In some control experiments, the coating was done with 100 μg/ml collagen instead. Blood was incubated with DiOC₆ (3) (5 μM) for 1 minute, or with Sensor Set 1 (SZ22-FITC (0.5 μg mL-1), Fg-Alexa Fluor 405 (60 μg mL-1), 2.2.9-Alexa Fluor 555 (1 μg mL-1) and AK4-Alexa Fluor 647 (1 μg mL-1)) or Set 2 (SZ22-FITC (0.5 μg mL-1), Annexin V-Pacific Blue (1 μg mL-1), MBC 370.2-Alexa Fluor 555 (1 μg mL-1) and PAC-1-Alexa Fluor 647 (1 μg mL-1)) for 10 min, and perfused through the channel. Thrombus formation was observed using a Leica DM IL LED microscope (camera: Leica DFC360 FX; objective lens: air, 20x; acquisition software: LAS X). No bleed-through between fluorescence channels was observed. Platelet autofluorescence was detected in 391-nm channel, which was subtracted when calculating signals.

In some experiments, different concentrations of aspirin (with 15 μg mL-1 defined as 2x) and/or clopidogrel (with 6 μg mL-1 defined as 2x) or ALB cocktail (1 U mL-1 apyrase, 100 mM MRS2179, 10 mM 2-MeSAMP, 10 μM indomethacin, 800 U mL-1 hirudin) were added into blood to inhibit platelet amplification loops.

Hill equation was used to derive IC50 of inhibitors:

$$Residue size = R + ( 100 - R ) / ( 1 + ( IC 50 / C ) \hat{} HillSlope ) \qquad ( 1 )$$

wherein C is the inhibitor concentration, R is the residue size when the effect of the inhibitor saturates, and HillSlope is a constant.

For subjects who were tested for multiple times, average values of these test results were used for data presentation and in statistical analyses.

Microfluidic laminar flow chamber assay. Reconstituted blood added with DiOC₆ (3) (10 μM) was perfused at different shear rates over straight channels pre-coated with VWFA1 or fibrinogen. After 5 min, fluorescent signals from platelets were recorded at 40 frame s-1.

Biomembrane force probe (BFP) and fluorescence BFP (fBFP). In a chamber filled with modified Tyrode's buffer +0.5% BSA (plus 1 mM Ca²⁺/Mg²⁺ when interrogating platelets with Fg beads), a streptavidin-coated glass probe bead was glued to the apex of a biotinylated RBC, which is aspirated by a micropipette to form an ultra-sensitive force probe. The probe bead was also coated with VWFA1 or Fg. On the opposing target side, a freshly isolated platelet was aspirated by a second micropipette, which was driven by a piezoelectric translator (Physical Instrument) to repeatedly bring the platelet in and out of contact with the bead to form adhesion events. The bead was monitored under an inverted microscope (IX83, Olympus) by a high-speed camera. A custom image analysis LabView (National Instrument) program tracks the bead position with 3 nm precision in real-time. The BFP spring constant k was determined by the suction pressure inside the probe pipette and the geometric parameters of the force transducer assembly.

For adhesion frequency assay, the platelet was repeatedly brought into contact with the probe bead for 2 seconds and retracted. Adhesion events were signified by the elongation of the RBC upon platelet retraction, which yielded a tensile force signal on the bead. Adhesion and non-adhesion events in 30 cycles were enumerated to calculate adhesion frequency, Pa. The effective avidity (A_{c}Kₐmᵣ) and affinity (A_{c}Kₐ) were derived by the following equation,

$${P}_{a} = 1 - exp \{ - {m}_{r} {m}_{l} {A}_{c} {K}_{a} \} \qquad ( 2 )$$

where mᵣ and m are the receptor and ligand surface densities derived from flow cytometry.

For force-clamp assay, contact time was shortened until achieving infrequent (˜20%) adhesion, which ensures that most (˜90%) of the adhesion events are mediated by single receptor-ligand bonds. Once an adhesion event was observed, the platelet would be held at a desired clamping force to wait for the bond to dissociate. Lifetime was determined as the time from the instant when the force reached the desired level to the instant of bond dissociation. The collected lifetimes were categorized into bins that cover successive force ranges. The average lifetime in each force bin was calculated to plot the “lifetime vs. clamping force” curve.

For fBFP, platelets were pre-loaded with Fura-2-AM and interrogated by VWFA1 beads with the force-clamp assay mode, but the contact time was kept at 2 seconds. Ratiometric imaging with a light source that alternates between 340 nm (to excite Ca²⁺-engaged Fura-2) and 380 nm (to excite Ca²⁺-free Fura-2) was used to measure the Ca²⁺ level in the aspirated platelet55. Signal intensity from the 340-nm channel was divided by that from the 380-nm channel and then normalized by the average value of the first 10 frames to derive the normalized Ca²⁺ level.

Flow cytometry assay. Platelet suspension was incubated with 2 μg mL-1 of HIP-8-Alexa Fluor 488, MBC370.2-Alexa Fluor 555, PAC-1-Alexa Fluor 647 or AK4-Alexa Fluor 647 for 10 min, diluted with Hepes-Tyrode buffer by 10 times, and immediately analyzed by flow cytometry.

Statistical Analysis. Statistical significance of the differences between two groups was determined by two-sided Student t-test. For the test of drug effects, multiple t-test assuming paired experimental design was used. For multi-group analysis, 1-way or 2-way ANOVA was used. When significant differences were shown, data was subjected to Turkey test for multiple comparisons. Regression slope test was used to assess whether the slope of a linear fitting is significantly non-zero. Spearman rank correlation coefficient and Kendall's tau correlation coefficient were also used to test whether a positive correlation exists between different readouts of the thrombus profile. P-values <0.05 were considered significant.

1. A method for characterizing a pathological condition or a thrombus regulating agent for effects on in vitro thrombogenesis comprising:
(i) applying shear stress to a blood sample in vitro forming a shear stress induced thrombus, the sample being from either a subject having or suspected of being at risk for pathological conditions related to thrombus formation or the sample is from a normal subject or a subject having a pathological condition related to thrombus formation that has been contacted with a thrombus regulating agent or an agent suspected of regulating thrombus formation or thrombus composition;
(ii) measuring levels of sensor targets including (a) integrin αIIbβ₃, (b) fibrinogen (Fg), (c) Von Willebrand Factor (VWF), (d) P-selectin, (e) conformationally extended integrin αIIbβ₃, (f) fully activated integrin αIIbβ₃, and (g) phosphatidylserine in the shear stress induced thrombus resulting in a thrombus profile;
(iii) generating an effect barcode for the pathological condition or the thrombus regulating agent based on differences in the thrombus profile relative to a reference, the effect barcode having a column for each sensor target level in (ii) above, each column including a positive, neutral, or negative effect of the pathological condition or thrombus regulating agent based on changes in the levels of the sensor targets.

2. The method of claim 1, wherein the column entry is a bar being at the top, middle or bottom of the column representing a positive, neutral, or negative effect, respectively.

3. The method of claim 1, wherein the column entry is expressed as + symbol, 0 or − symbol representing a positive, neutral, or negative effect, respectively.

4. A method for thrombus profiling comprising,
(i) applying a blood sample to a shear stress thrombosis simulation device forming an in vitro thrombus;
(ii) contacting the in vitro thrombus with a plurality of sensor agents, the sensor agents comprising a detectable label and a component specific binding moiety and quantifying binding of the plurality of sensor agents to the in vitro thrombus determining each sensor agent result;
(iii) generating a thrombus profile by characterizing the binding of each sensor agent, forming a thrombus profile comprising binding results for each sensor agent in an ordered readout.

5. The method of claim 4, further comprising characterizing the functional effect of an anti-thrombotic agent based on changes in the thrombus profile.

6. The method of claim 4, further testing blood of a subject having a predetermined sensor agent profile for a first condition or a first anti-thrombotic agent after the subject is exposed to a second anti-thrombotic treatment or a second anti-thrombotic agent for assessing efficacy and safety of the second anti-thrombotic treatment or the second anti-thrombotic agent, or select an optimal treatment regimen for the subject.

7. The method of claim 4, wherein the binding moieties comprise a (a) conformation independent integrin αIIbβ₃ binding moiety, (b) fibrinogen (Fg) binding moiety, (c) Von Willebrand Factor (VWF) binding moiety, (d) P-selectin binding moiety, (e) conformationally extended integrin αIIbβ₃ binding moiety, (f) fully activated integrin αIIbβ₃ binding moiety, and (g) phosphatidylserine binding moiety.

8. The method of claim 7, wherein (a) the conformation independent integrin αIIbβ₃ binding moiety is a SZ22 antibody, (b) the fibrinogen (Fg) binding moiety is fibrinogen, (c) Von Willebrand Factor (VWF) binding moiety is a 2.2.9 antibody, (d) the P-selectin binding moiety is a AK4 antibody, (e) the conformationally extended integrin αIIbβ₃ binding moiety is a MBC 370.2 antibody, (f) the fully activated integrin αIIbβ₃ binding moiety is a PAC-1 antibody, and (g) the phosphatidylserine binding moiety is Annexin V.

9. A method for generating a thrombotic profile comprising:
(ii) contacting an in vitro thrombus with a plurality of sensor agents, the sensor agents comprising a detectable label and a component specific binding moiety, the binding moieties comprising a (a) conformation independent integrin αIIbβ₃ binding moiety, (b) fibrinogen (Fg) binding moiety, (c) Von Willebrand Factor (VWF) binding moiety, (d) P-selectin binding moiety, (e) conformationally extended integrin αIIbβ₃ binding moiety, (f) fully activated integrin αlbβ₃ binding moiety, and (g) phosphatidylserine binding moiety;
(iii) quantifying binding of the plurality of sensor agents to the in vitro thrombus determining each sensor agent result; and
(iv) generating a thrombotic profile of the subject by characterizing the binding of each sensor agent as either increased, no change, or decreased as compared to a reference, forming a thrombotic profile of a subject comprising binding results for each sensor agent in an ordered readout.

10. The method of claim 9, wherein (a) the conformation independent integrin αIIbβ₃ binding moiety is a SZ22 antibody, (b) the fibrinogen (Fg) binding moiety is fibrinogen, (c) Von Willebrand Factor (VWF) binding moiety is a 2.2.9 antibody, (d) the P-selectin binding moiety is a AK4 antibody, (e) the conformationally extended integrin αIIbβ₃ binding moiety is a MBC 370.2 antibody, (f) the fully activated integrin αIIbβ₃ binding moiety is a PAC-1 antibody, and (g) the phosphatidylserine binding moiety is Annexin V.

11. A kit for performing a thrombus profile assay comprising (a) a conformation independent intgrin αIIbβ₃ binding moiety, (b) fibrinogen (Fg) binding moiety, (c) Von Willebrand Factor (VWF) binding moiety, (d) P-selectin binding moiety, (e) conformationally extended integrin αIIbβ₃ binding moiety, (f) fully activated integrin αIIbβ₃ binding moiety, and (g) phosphatidylserine binding moiety.

12. The kit of claim 11, further comprising conjugation reagents for labeling the binding moieties.

13. The kit of claim 11, further comprising a microfluidic device configured for inducing shear stress thrombus formation.
